(19) United States
(12) Patent Application Publication
(10) Pub. No.: US 2022/0180963 A1
(43) Pub. Date: Jun. 9, 2022

(54) METHOD FOR SELECTING PATIENT SPECIFIC THERAPY

(71) Applicant: YISSUM RESEARCH DEVELOPMENT COMPANY OF THE HEBREW UNIVERSITY OF JERUSALEM LTD., Jerusalem (IL)

(72) Inventors: Nataly KRAVCHENKO-BALASHA, Kfar Uriya (IL); Raphael David LEVINE, Jerusalem (IL); Efrat FLASHNER-ABRAMSON, Givat Yearim (IL)

(73) Assignee: YISSUM RESEARCH DEVELOPMENT COMPANY OF THE HEBREW UNIVERSITY OF JERUSALEM LTD., Jerusalem (IL)

(21) Appl. No.: 17/051,363

(22) PCT Filed: Apr. 29, 2019
(86) PCT No.: PCT/IL2019/050474

§ 371 Date: Oct. 28, 2020

Related U.S. Application Data
(60) Provisional application No. 62/664,202, filed on Apr. 29, 2018.

(51) Int. Cl. G16B 5/00; C12Q 1/6886

(52) U.S. Cl. CPC G16B 5/00; C12Q 2600/158; C12Q 2600/106; C12Q 1/6886

(57) ABSTRACT

Methods of identifying a druggable target in a subject suffering from cancer comprising determining at least one unbalanced process in the subject's expression data and selecting at least one gene and/or protein from the at least one unbalanced process wherein a drug that targets that gene or protein is known.

### CROSS-REFERENCE TO RELATED APPLICATIONS

This application claims the benefit of priority of U.S. Provisional Patent Application No. 62/664,202 titled “METHOD FOR SELECTING PATIENT SPECIFIC THERAPY”, filed Apr. 29, 2018, the contents of which is incorporated herein by reference in its entirety.

### FIELD OF INVENTION

The present invention is in the field of personalized cancer therapy.

### BACKGROUND OF THE INVENTION

Cancer is a complex disease, characterized by a malfunctioning of signaling networks. Aberrant signaling events play key roles in the maintenance and progression of tumors. This understanding has spurred the development of targeted therapies, specifically aimed at proteins that transduce signals through the defective pathways. However, though targeted anti-cancer therapy initially showed considerable promise, it soon became clear that single targeted agents seldom suffice to induce complete tumor remission. The molecular variability among different tumors, referred to as inter-tumor heterogeneity, greatly complicates the prediction of the tumor's response to the treatment, and therefore the designation of the appropriate therapy.

A plethora of analytical methods that address the complex nature of protein networks have been developed: Bayesian methods, based on elucidating the relationships between a few genes at a time; reverse-engineering algorithms, based on chemical kinetic-like differential equations; and multivariate statistical methods that include clustering methods, principal component analysis, singular value decomposition and meta-analysis. These methods have significantly progressed the fields of computational analysis and cancer research. However, the majority of aggressive tumors still do not respond well to therapy.

To tackle tumor heterogeneity, personalized cancer therapy has been set at the frontline of cancer research and cancer therapy. Nonetheless, the emerging fields of personalized therapy and precision therapy still do not address individual tumors, but rather aim to mainly cluster tumors into groups according to similarity. However, tumors that are classified as similar according to the expression levels of certain oncogenes can eventually demonstrate divergent responses to treatment. This implies that the information gained from the identification of tumor-specific biomarkers is still not sufficient. There is a great need to personalizing cancer therapy, so that the drugs best suited for treating each individual patient are actually given to that patient.

### SUMMARY OF THE INVENTION

The present invention provides methods of identifying a druggable target in a subject suffering from cancer comprising determining at least one unbalanced process in the subject's expression data and selecting at least one gene and/or protein from the at least one unbalanced process wherein a drug that targets that gene or protein is known.

According to a first aspect, there is provided a method of identifying a druggable target, in a subject suffering from cancer, the method comprising,
a. receiving expression data from the subject;
b. adding the subject's expression data to a first composite cancer-expression data set to produce a second composite cancer-expression data set;
c. determining within the second composite cancer-expression data set at least one unbalanced process, wherein the determining comprises performing thermodynamic-based analysis;
d. identifying within the subject's expression data at least one of the at least one unbalanced processes within the second composite cancer-expression data set; and
e. selecting at least one gene and/or protein from the at least one unbalanced process within the subject's expression data for which a drug that targets the gene or protein is known;
   thereby identifying a druggable target in a subject suffering from cancer.

According to some embodiments, the expression data is protein expression data or mRNA expression data. According to some embodiments, the receiving expression data comprises receiving a biological sample from the subject and performing high-throughput sequencing on the sample.

According to some embodiments, the biological sample is a blood sample or a tumor biopsy.

According to some embodiments, the method further comprises normalizing the subject's expression data with a composite healthy-expression data set or with a composite healthy and cancer-expression data set.

According to some embodiments, determining at least one unbalanced process comprises determining over and under expressed genes and/or proteins as compared to their expression in a balanced process. According to some embodiments, determining at least one unbalanced process comprises assembling expressed genes and/or proteins within the second data set into networks.

According to some embodiments, the assembling is performed using functional interactions according to the STRING database.

According to some embodiments, the thermodynamic-based analysis comprises surprisal analysis.

According to some embodiments, the first composite cancer-expression data set comprises data from at least 1 type of cancer. According to some embodiments, the different types of cancer are selected from lymphoma, bladder cancer, gastric cancer, colorectal cancer, kidney cancer, ovarian cancer, endometrial cancer, lung cancer, head and neck cancer, brain cancer and breast cancer.

According to some embodiments, the first composite cancer-expression data set comprises data from at least 10 samples.

According to some embodiments, the selected at least one unbalanced process is selected from Table 1.

According to some embodiments, the at least one gene or protein is over or under expressed in the subject's expression data. According to some embodiments, the at least one gene or protein is a known cancer regulatory gene or protein. According to some embodiments, the at least one gene or protein is selected from Table 1 and Table 3.

According to some embodiments, the methods of the invention further comprise administering to the subject the known drug.

According to another aspect, there is provided a method for patient-specific cancer treatment, the method comprising,
a. identifying at least one druggable target specific to the patient using a method of the invention; and
b. administering to the subject at least one drug that targets the at least one druggable target,

thereby providing patient-specific cancer treatment.

According to some embodiments, the method further comprises repeating a method of the invention after a period of treatment with the at least one drug to determine at least one new druggable target. According to some embodiments, the method further comprises administering the at least one new druggable target. According to some embodiments, the at least one drug is selected from Table 3.

According to another aspect, there is provided a computer program product for identifying a druggable target, in a subject suffering from cancer, comprising a non-transitory computer-readable storage medium having program code embodied thereon, the program code executable by at least one hardware processor to
a. receive expression data from the subject;
b. add the subject's expression data to a first composite cancer-expression data set to produce a second composite cancer-expression data set;
c. determine within the second composite cancer-expression data set at least one unbalanced processes, wherein the determining comprises performing thermodynamic-based analysis;
d. identify within the subject's expression data at least one of the at least one unbalanced processes within the second composite cancer-expression data set; and
e. provide an output of at least one gene and/or protein from the at least one unbalanced process within the subject's expression data for which a drug that targets the gene or protein is known.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-F: Identifying the steady state in cancer patients and integrating biological datasets to study inter-tumor heterogeneity. (1A) A dot plot showing amplitudes of balanced process (λ₀) for patients with lymphoma (Ly; patients 1-130), bladder cancer (Bl; patients 131-223), healthy (H; patients 224-244), gastric cancer (Ga; patients 245-355), colorectal cancer (Co; patients 356-507) and breast cancer (Br; patients 508-527) before and after normalization. (1B) An exemplary dot plot from gastric cancer is shown. The abscissa is the Gᵢ₀(n) values after normalization and the ordinate is the values before normalization of gastric cancer dataset—Gᵢ₀. The transcript composition of the steady state remains similar before and after the normalization, as can be seen by the high correlation between Gᵢ₀ and Gᵢ₀(n). For all the other five samples the correlation was 0.9 or higher. (1C) Dot plots showing the partial error, representing variance in expression data for patient 193. Partial errors for different number of constraints are presented: (first plot) includes only α=0; the steady state (sts); (second plot) steady state and the first 3 unbalanced processes, α=0, 1, 2, 3; (third plot) the steady state and the first 5 unbalanced processes; (fourth plot) the steady state and the first 8 unbalanced processes; (fifth plot) the steady state and the first 10 unbalanced processes; (six plot) the steady state and the first 12 unbalanced processes; (seventh plot) the steady state and the first 15 unbalanced processes. (1D) Line graphs of R² values (left panel) and root mean square values (right panel) for patient 193. (1E) The same as in 1D, but for patient 510. (1F) The same as in 1C, but for patient 510.

FIGS. 2A-D: (2A) A line graph of weights of all the transcripts for unbalanced process 1. (2B) A bar graph showing the frequency of the unbalanced processes in every cancer type. −100 denotes 100% with negative amplitude, 100 denotes 100% with positive amplitude. Note that at least one unbalanced process is common to all patients of a given cancer type. For example, processes 1+ and 2+ were found in all patients of lymphoma (Ly); 1− was found in all patients of bladder cancer (Bl). Furthermore, most of the processes each appear in at least two types of cancer. For example, process 3 (+ or −) was found in all cancer types (Ly, Bl, Co (colorectal), Ga (gastric) and Br (breast)). (2C) A chart of patient-specific combinations of unbalanced process. Two selected patients from each cancer type are shown. (2D) A bar chart of patient-specific combinations of unbalanced processes.

FIGS. 3A-C: Similar gene expression levels in different patients may be attributed to different unbalanced processes. Two bladder cancer patients, along with the six processes that characterize those patients, were selected to demonstrate how similar expression levels can be attributed to different unbalanced processes. (3A) A bar chart showing the fold changes of five selected bladder cancer-associated oncogenes. NFKBIA, PD-L1, CD44, EGFR, and PLAU were upregulated in both patients relative to their median expression levels across the 506 patients. (3B) A chart of patient-specific combinations of unbalanced processes. Patient 164 harbors processes 1, 2, 5, 9 and 10. Patient 172 harbors unbalanced processes 1, 2, 5, and 7. (3C) Transcript network charts showing the influences of these unbalanced processes on 19 selected oncogenes. Black color denotes upregulation; white color denotes downregulation; gray color denotes no change due to the process. Functional connections are according to STRING database.

FIGS. 4A-G: The 13 unbalanced processes fully characterize 39 pancreatic patients. (4A) A line graph showing R² values calculated for all pancreatic patients by plotting the natural logarithm of the experimental data ln(Xᵢ(k)) vs ΣG_{iα}λ_{α}(k) for different values of α. The value of R² approaches 1 as more unbalanced processes are added to the calculation. (4B) A line graph showing the 36% of the pancreatic patients fully characterized by the first 12 unbalanced processes. 4 selected patients are shown. The gray box highlights that the addition of the 13^{th} constraint had no significant effect on the R² value for these patients. (4C) A line graph showing the 64% of the pancreatic patients found to harbor an additional pancreatic-specific constraint, unbalanced process 13, which did not appear in the analysis of the 506 original patients. The gray box highlights that the addition of the 13^{th} constraint is significant for these patients. (4D) A chart of the frequency of the unbalanced processes in the 39 pancreatic cancer patients. (4E) A bar chart of the combinations of unbalanced processes identified in the pancreatic cancer dataset. (4F-G) Dot plots comparing the 12 processes found before and after addition of pancreatic cancer dataset. “ori” denotes the original analysis and “val” denotes the values after adding the pancreatic cancer dataset. Both the (4F) amplitudes of the processes and (4G) weights of the transcripts are compared.

FIG. 5: A diagram of the protocol for surprisal analysis.

FIGS. 6A-C: (6A) Network diagrams of protein-protein interactions in the 17 unbalanced processes. ppAkt=Akt phosphorylated on Thr308 and on Ser473; ppEGFR=EGFR phosphorylated on Tyr1068 and on Tyr1173. Black circles indicate genes with positive G_{iα} values; white circles indicate genes with negative G_{iα} values. (6B) A graph of values from a given process α=1. Shown are sorted values of Gᵢ₁, which represent the degree of participation of every protein i in the unbalanced process α=1. The gray box represents threshold values. Proteins with Gᵢ₁>0.1 or Gᵢ₁<−0.1 (which are not contained in the gray box and form the top and bottom “tails” of the distribution) were considered to participate the most in the unbalanced process α=1. These proteins were used to build a functional network using STRING database, presented in 6A. (6C) Dot plots comparing the theoretical expression level of pEGFR in a given tumor versus the experimental expression level when (upper left panel) only the first, most significant unbalanced process was taken into account (n=1); (upper right panel) the 10 most significant unbalanced processes were taken into account (n=10); (lower left panel) the 17 most significant unbalanced processes were taken into account (n=17); (lower right panel) the 21 most significant unbalanced processes were taken into account (n=21).

FIGS. 7A-B: Patient diagnoses remain essentially the same when a smaller matrix is analyzed. (7A) Line graphs for 3 patients showing λ_{α} values calculated with the large (3467 samples) and the small (1100) matrices. (7B) Dot plot of Gᵢ₁ values obtained from the analysis of the small matrix plotted against Gᵢ₁ values obtained from the analysis of the original matrix.

FIGS. 8A-B: EGFR signaling pathway undergoes significant rewiring in different unbalanced processes. (8A) A bar chart showing G_{iα} values—the weight of participation of the activated form of EGFR (pY(1068)EGFR or pY(1173)EGFR, in every unbalanced process α. The dashed lines mark threshold limits (for details see Methods). (8B) A chart of 3 representative patients chosen in order to demonstrate the significant rewiring of the EGFR signaling pathway in different unbalanced networks. Note: Active unbalanced processes were assigned a significant amplitude (marked gray for negative and black for positive), whereas inactive unbalanced processes (marked white) were assigned an insignificant amplitude (see Methods).

FIGS. 9A-E: A two-dimensional representation of the tumor imbalances masks the patient-explicit network structure. (9A-C) Dot plots of the amplitudes of the three most significant unbalanced processes, (9A) λ₁(k), (9B) λ₂(k), and (9C) λ₃(k), plotted against each other in pairs in order to examine the information that these plots can provide. (9D) A bar chart of the amplitudes, λ_{α}(k), of the 17 significant unbalanced processes were plotted for the 215 glioblastoma multiforme (GBM) patients. The gray box marks threshold limits. (9E) Bar charts of the same analysis as in 9D, but for the other 10 cancer types.

FIGS. 10A-B: The majority of tumors are characterized by rare barcodes. (10A) A bar chart showing that 452 unique barcodes characterize the entire population of 3467 tumors. The different barcodes were sorted according to their frequency in the entire population of tumors. The dashed box shows the 16 most abundant barcodes. The rare barcodes (376 barcodes which characterize only 5 tumors or less) are highlighted in grey. (10B) Bar charts showing the barcodes for each of the tumor types, including the collection of unique barcodes that characterize each specific tumor type. The rare barcodes (376 barcodes which characterize only 5 tumors or less) are highlighted in grey.

FIG. 11: Mapping the patients into a 17-dimensional data space. A bar chart mapping the 16 most abundant barcodes, each appearing in 1% or more of the patients (at least 35 patients). Each tumor-specific barcode identified is mapped to specific coordinates in the 17-dimensional space, according to the active unbalanced processes and the sign of their amplitude. The graph presents a two-dimensional form of this concept—each column in the graph represents multiple patients that were mapped to the same coordinates in the 17-dimensional space. Y axis represents the percentage of patients in each cancer type (thus the values of the bars may exceed 100%. For example, 19% of bladder cancer patients harbored barcode 10). Note: Active unbalanced processes are such that exhibit λ*_{α}(k)=±1 (marked black/gray), whereas inactive unbalanced processes are such that exhibit λ*_{α}(k)=0 (marked white).

FIG. 12: Prediction of patient-specific combination therapy. Chart of 5 patients, their unbalanced processes, their treatable hubs and possible treatment regimes. Note: Active unbalanced processes are such that exhibit λ*_{α}(k)=±1 (marked gray/black), whereas inactive unbalanced processes are such that exhibit λ*_{α}(k)=0 (marked white).

FIG. 13A-F: Workflow of our information-theoretic approach. A diagram of the workflow for the analysis of the invention. Following acquisition of samples (13A), a dataset is constructed using proteomics techniques (13B). Surprisal analysis is then utilized (13C) in order to uncover the complete patient-specific protein network structure, comprising balanced and unbalanced molecular processes, in which all molecules undergo coordinated changes in expression. Next, a patient-specific barcode is constructed, indicating the set of significant unbalanced processes that influence the specific tumor (13D), and the tumor-specific unbalanced network is examined, aiming to identify and verify experimentally the major hubs whose blockage is suggested to lead to a collapse of the unbalanced network (13E). Finally, a tumor-specific combination of targeted therapies is tailored to every patient (13F).

FIG. 14A-I. Experimental validation of the approach (14A, 14D, 14G) Diagrams of signaling signatures of MDA-MB-231, MDA-MB-468 and MCF7 cells, according to SA based analysis. Schematic figures of each unbalanced process are shown. Drug predictions are indicated for each cell line, including the processes that they are predicted to target. (14B, 14E, 14H) Bar charts of survival rates in response to different treatments (as measured by methylene blue). For each cell line the combination of drugs predicted to target the complete unbalanced signaling signature was tested (marked with an asterix), as well as combinations that were predicted to only partially target the unbalanced signaling flux, each drug alone and taxol. For MDA468 a triple combination was tested as well (marked with two asterix signs, shown in the inset in FIG. 14E). In this case the signaling signature consists of 5 unbalanced processes (see FIG. 14D), therefore we postulated that these cells may require an additional drug that will enhance the inhibition of the imbalance, as is indeed shown. Moreover, the predicted and highly efficient combination for MDA-MB-231, Trametinib and 2-DG, was significantly less efficient in killing of MDA-MB-468, pointing to the specificity and the selectivity of our predicted treatments (14C, 14F, 14I) Western blot analyses of the cells following different treatments. Stain-free imaging was used to ensure equal loading. In all 3 cases, our predicted drug combinations induced high levels of PARP cleavage relative to other treatments. Bar chart inserts show quantified relative expression of the investigated protein.

FIG. 15. The predicted therapy works more efficiently than Taxol used in clinics. 7 week-old SCID mice were orthotopically injected with MDA231 cells (2.5×10⁶ in 100 □l of DMEM:cultrex 1:1) in the left fourth mammary fat pad. Treatments were initiated once tumors reached a volume of 100 mm³ and lasted 3 weeks. Combination treatment (0.5 mg/kg trametinib (T) and 50 mg/kg 2-DG) and vehicle (0.5% hydroxypropylmethyl cellulose+0.2% Tween-80) were administered orally 5 d/week. Taxol (20 mg/kg) was administered intraperitoneally once a week. The initial groups were 9 mice/group. Some of the mice were euthanized when they reached the humane endpoint as indicated by the ethical committee (excessive tumor volume or skin necrosis over the tumor). Standard error and statistically significant p value of t-test are shown in the graph.

### DETAILED DESCRIPTION OF THE INVENTION

The present invention, in some embodiments, provides methods of identifying a druggable target in a subject suffering from cancer, comprising determining at least one unbalanced process (i.e. altered network) in the subject's expression data and selecting at least one gene and/or protein from the at least one unbalanced process, wherein a drug that targets that gene or protein is known. A computer program product for doing same is also provided.

The invention is based on the surprising finding that cancers with similar biomarker expression, can harbor unique unbalanced processes. Cancers are often grouped by the source of the cancer and a handful of potentially informative biomarkers. And yet cancers from the same source and with similar biomarker expression can have radically different responses to treatment. The inventors have found that this is due, at least in part, to different unbalanced processes within in the tumor, and by identifying the unbalanced processes suitable treatment can be selected for each individual tumor.

By a first aspect, there is provided a method of identifying a druggable target, in a subject suffering from cancer, the method comprising,
a. receiving expression data from the subject;
b. adding the subject's expression data to a first composite cancer-expression data set to produce a second composite cancer-expression data set;
c. determining within the second composite cancer-expression data set at least one unbalanced process, wherein the determining comprises performing thermodynamic-based analysis;
d. identifying within the subject's expression data at least one of the at least one unbalanced processes within the second composite cancer-expression data set; and
e. selecting at least one gene and/or protein from the at least one unbalanced process within the subject's expression data for which a drug that targets the gene or protein is known;
   thereby identifying a druggable target in a subject suffering from cancer.

By another aspect, there is provided a method of identifying a druggable target, in a subject suffering from cancer, the method comprising,
a. receiving expression data from the subject;
b. adding the subject's expression data to a first composite cancer-expression data set to produce a second composite cancer-expression data set;
c. determining at least one unbalanced processes within the subject's expression data, wherein the determining comprises performing thermodynamic-based analysis on the second composite cancer-expression data set; and
d. selecting at least one gene and/or protein from the at least one unbalanced process for which a drug that targets the gene or protein is known;
   thereby identifying a druggable target in a subject suffering from cancer.

In some embodiments, the methods of the invention are performed ex vivo. In some embodiments, the methods of the invention are computerized methods. In some embodiments, the methods of the invention are performed on a computer. In some embodiments, the data provided, and the output of the method are embodied in electronic files.

As used herein, a “druggable target” refers to any gene or protein whose expression or function can be modified by administration of a drug. Potential drugs can be selected from any known drug list, or database, including but not limited to the FDA approved drug list, the National Cancer Institute drug list (cancer.gov/about-cancer/treatment/drugs), and drugs.com. In some embodiments, the drug effects only the druggable target. In some embodiments, the drug effects more than one target including the druggable target.

In some embodiments, the cancer is any cancer. In some embodiments, the cancer is a solid cancer. In some embodiments, the cancer is a blood cancer. In some embodiments, the cancer is a solid cancer or a blood cancer. In some embodiments, the cancer is selected from lymphoma, bladder cancer, gastric cancer, colorectal cancer, and breast cancer. In some embodiments, the cancer is selected from lymphoma, bladder cancer, gastric cancer, colorectal cancer, pancreatic cancer and breast cancer. In some embodiments, the cancer is selected from breast cancer, colon cancer, rectal cancer, kidney cancer, ovarian cancer, endometrial cancer, lung cancer, bladder cancer, and brain cancer. In some embodiments, the cancer is selected from breast cancer, colon cancer, rectal cancer, kidney cancer, ovarian cancer, endometrial cancer, lung cancer, bladder cancer, lymphoma, gastric cancer, colorectal cancer and brain cancer. In some embodiments, the cancer is selected from breast cancer, colon adenocarcinoma, rectal adenocarcinoma, kidney renal cell carcinoma, ovarian cancer, endometrial carcinoma, lung squamous cell carcinoma, bladder carcinoma, and glioblastoma multiforme. In some embodiments, the brain cancer is glioblastoma multiforme. In some embodiments, the cancer is adenocarcinoma. In some embodiments, the cancer is carcinoma.

In some embodiments, the expression data is embodied in an electronic file. In some embodiments, the expression data is protein expression data. In some embodiments, the expression data is proteomics expression data. In some embodiments, the expression data is mRNA expression data. In some embodiments, the expression data is transcriptional expression data. In some embodiments, the expression data is protein or mRNA expression data. In some embodiments, the expression data is proteomics or transcriptional expression data. In some embodiments, the expression data is proteomics and transcriptional expression data. In some embodiments, the expression data is from massively parallel sequencing or an equivalent sequencing technique. In some embodiments, the expression data is from a proteomics analysis.

In some embodiments, receiving expression data comprises receiving a biological sample from the subject. In some embodiments, high-throughput sequencing is performed on the sample. In some embodiments, the sequencing is nucleotide sequencing. In some embodiments, the sequencing is protein sequencing. In some embodiments, the sequencing is nucleotide or protein sequencing. In some embodiments, the sequencing is nucleotide and protein sequencing.

In some embodiments, the biological sample is a sample of the cancer. In some embodiments, the sample of the cancer is a tumor biopsy. In some embodiments, the sample of the cancer is a liquid biopsy. In some embodiments, the biological sample is a blood sample from the subject. In some embodiments, the biological sample is a blood sample or a sample of the cancer.

As used herein, a “composite cancer expression data set” refers to expression data from more than one cancer sample. In some embodiments, the first and second cancer expression data sets are embodied in digital files. In some embodiments, the composite expression data set is a database of cancer expression profiles. In some embodiments, the data set comprises data from at least 2, 5, 10, 20, 30, 40, 50, 100, 200, 300, 400, 500, 600, 700, 800, 900, 1000, 1500, 2000, 2500, 3000, 3500, 4000, 4500, 5000 or 5500 samples. Each possibility represents a separate embodiment of the invention. In some embodiments, the data set comprises data from at least 10 samples. In some embodiments, the data set comprises data from at least 1, 2, 3, 5, 10, 15, 20, or 25 different types of cancer. Each possibility represents a separate embodiment of the invention. In some embodiments, the data set comprises data from at least 5 different types of cancer. In some embodiments, the data in the data set is all from the same type of cancer. In some embodiments, the data is all from triple negative breast cancer. In some embodiments, the data set comprises expression data from at least one healthy sample.

In some embodiments, the method further comprises normalizing the subject's expression data. In some embodiments, the normalizing is performed with a composite healthy-expression data set. As used herein, a “composite healthy-expression data set” refers to expression data from more than one healthy sample. In some embodiments, the normalizing is performed with a composite healthy and cancer-expression data set. The inventors have demonstrated previously that healthy and cancerous patients have a common baseline of balanced processes (Kravchenko-Balasha et al., 2012, On a fundamental structure of gene networks in living cells, PNAS, 109 (12) 4702-07), thus the normalizing can be performed with a mixed data set. In some embodiments, the normalizing can be done with a composite healthy-expression data set or with a composite healthy and cancer-expression data set.

Normalization of data is well known in the art and any method or algorithm for normalization may be employed. In some embodiments, the normalization is performed as described herein. In some embodiments, the normalization is according to the median expression values. In some embodiments, the normalization comprises using equation [2] as disclosed herein.

As used herein, a “balanced process” refers to a network of genes/proteins that exists in the sample at maximal entropy or thermodynamic equilibrium. Thus, a balanced process is a network in a balanced state. As used herein, an “unbalanced process” refers to a network of genes/proteins that deviates from the balanced state. This is a network that deviates from thermodynamic steady state. In some embodiments, a process is a signaling network. In some embodiments, a process is a signaling pathway. In some embodiments, a process is a functional pathway. In some embodiments, a process is a functional network.

In some embodiments, determining at least one unbalanced process comprises determining over and under expressed genes and/or proteins in each sample's expression data. In some embodiments, the over and under expression is as compared to a control data set. In some embodiments, the over and under expression is as compared to the average expression in the first or second data set. In some embodiments, the over and under expression is as compared to the median expression in the first or second data set. In some embodiments, the over and under expression is as compared to other genes/proteins within an unbalanced process. In some embodiments, the over and under expression is as compared to other genes/proteins within the process being examined. A skilled artisan will appreciate that when a process is examined for being balanced or unbalanced a single gene/protein can be determined to be over or under expressed relative to the expression of the other genes/proteins of the process. In some embodiments, determining at least one unbalanced process comprises determining within the second composite cancer-expression data set all unbalanced processes and identifying at least one of those unbalanced processes that is within the subject's expression data.

In some embodiments, determining at least one unbalanced process comprises assembling expressed genes and/or proteins into networks. In some embodiments, the networks are assembled from genes/proteins from the first data set. In some embodiments, the networks are assembled from genes/proteins from the second data set. In some embodiments, the networks are assembled from genes/proteins from the first data set and/or the second data set. In some embodiments, the networks are functional networks. In some embodiments, the assembling is performed using functional interactions. In some embodiments, the function interactions are according to the STRING database.

In some embodiments, the thermodynamic-based analysis is an information theoretical analysis. In some embodiments, the thermodynamic-based analysis is a thermodynamic-based information theoretical analysis. In some embodiments, the thermodynamic-based analysis comprises surprisal analysis. In some embodiments, the thermodynamic-based analysis is surprisal analysis. As used herein, “surprisal analysis” refers to an analysis technique that determines thermodynamic and entropic balanced and unbalanced states in a system. In some embodiments, the surprisal analysis comprises the analysis described herein. In some embodiments, the surprisal analysis comprises using equation [1].

In some embodiments, at least one unbalanced process is identified in a subject's expression data. In some embodiments, all unbalanced processes are identified in a subjects' expression data. In some embodiments, all unbalanced processes that exist in the second data set and exist in the subject's expression data are identified. In some embodiments, the at least one unbalanced process is selected from Table 1. In some embodiments, the methods of the invention comprise assigning to a sample a barcode. In some embodiments, the barcode indicates the unbalanced processes in the sample. In some embodiments, the barcode indicates the status of all processes in the sample.

In some embodiments, the selected at least one gene or protein is over or under expressed in the subject's expression data. In some embodiments, the selected at least one gene or protein is the most over or under expressed gene/protein in the identified at least one unbalanced process. In some embodiments, the selected at least one gene or protein is a hub gene/protein of the identified at least one unbalanced process. As used herein, a “hub gene/protein” refers to a gene/protein that has a large number of biologically-relevant to cancer protein-protein connections in a process. In some embodiments, the selected at least one gene or protein is a central protein of the process. the selected at least one gene or protein is a known cancer regulatory gene/protein. In some embodiments, the selected at least one gene or protein has a known drug that modulates the gene/protein's function and/or expression. In some embodiments, the selected at least one gene or protein is selected from Table 1. In some embodiments, the selected at least one gene or protein is selected from Table 3. In some embodiments, the selected at least one gene or protein is selected from Table 1 and/or Table 3.

In some embodiments, the methods of the invention further comprise administering the known drug to the subject. In some embodiments, the known drug is any anticancer drug. In some embodiments, the known drug is selected from Table 3. In some embodiments, the known drug effects the target gene/protein. In some embodiments, the known drug effects the target gene/protein such that it corrects the imbalance in the process. Examples of this would be a protein that is over expressed and a drug that reduces expression, or a protein that is under expressed and drug that induces expression. In some embodiments, the known drug brings the unbalanced process into balance.

As used herein, the terms “administering,” “administration,” and like terms refer to any method which, in sound medical practice, delivers a composition containing an active agent to a subject in such a manner as to provide a therapeutic effect. Suitable routes of administration can include, but are not limited to, oral, parenteral, subcutaneous, intravenous, intramuscular, or intraperitoneal.

The dosage administered will be dependent upon the age, health, and weight of the recipient, kind of concurrent treatment, if any, frequency of treatment, and the nature of the effect desired.

By another aspect, there is provided a method for patient-specific cancer treatment, the method comprising,
a. identifying at least one druggable target specific to the patient using a method of the invention; and
b. administering to the subject at least one drug that targets the at least one druggable target,
   thereby providing patient-specific cancer treatment.

In some embodiments, the at least one drug is a known drug. In some embodiments, the at least one drug is any anticancer drug. In some embodiments, the at least one drug is selected from Table 3.

In some embodiments, the method further comprises repeating the method of identifying a druggable target after a period of treatment with the at least one drug. In some embodiments, repeating the method determines if the administered drug has returned the unbalanced process to a balanced state. In some embodiments, repeating the method determines at least one new druggable target. In some embodiments, the method further comprises administering the new at least one druggable target. In some embodiments, the method is repeated indefinitely. In some embodiments, the method is repeated until the subject is cancer free.

By another aspect, there is provided a computer program product for identifying a druggable target, in a subject suffering from cancer, comprising a non-transitory computer-readable storage medium having program code embodied thereon, the program code executable by at least one hardware processor to
a. receive expression data from the subject;
b. add the subject's expression data to a first composite cancer-expression data set to produce a second composite cancer-expression data set;
c. determine within the second composite cancer-expression data set at least one unbalanced processes, wherein the determining comprises performing thermodynamic-based analysis;
d. identify at least one unbalanced processes within the subject's expression data; and
e. provide an output of at least one gene and/or protein from the at least one unbalanced thermodynamic process for which a drug that targets the gene or protein is known.

By another aspect, there is provided a computer program product for identifying a druggable target, in a subject suffering from cancer, comprising a non-transitory computer-readable storage medium having program code embodied thereon, the program code executable by at least one hardware processor to
a. receive expression data from the subject;
b. add the subject's expression data to a first composite cancer-expression data set to produce a second composite cancer-expression data set;
c. determine at least one unbalanced processes within the subject's expression data, wherein the determining comprises performing thermodynamic-based analysis; and
d. provide an output of at least one gene and/or protein from the at least one unbalanced thermodynamic process for which a drug that targets the gene or protein is known.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention, include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Methods

Surprisal analysis. Surprisal analysis is a thermodynamic-based information-theoretic approach. The analysis is based on the premise that biological systems reach a balanced state when the system is free of constraints. However, when under the influence of environmental and genomic constraints, the system is prevented from reaching the state of minimal free energy, and instead reaches some steady state which is higher in free energy.

The analysis takes as an input the expression levels of macromolecules, such as genes, transcripts, or proteins, and utilizes the following equation:

$$ln {X}_{i} ( k ) = ln {X}_{i}^{o} ( k ) - {\sum }_{α} {G}_{i α} {λ}_{α} ( k ) \qquad [ 1 ]$$

For every molecule i in the sample k, the logarithm of its experimental expression level, ln Xᵢ(k), is broken down to the logarithm of its expression level at the balanced state, ln Xᵢ⁰(k), and the sum of deviations from this level due to the different constraints that operate on the system,

$${\sum }_{α} {G}_{i α} {λ}_{α} ( k ) .$$

Each constraint is associated with an unbalanced biological process that operates on the system. These processes are indexed by α=1, 2, 3 . . . , such that the significance of the process decreases with increasing index.

Mathematically, the algorithm is based on the construction of a covariance matrix of the logarithm of the expression levels, and the usage of SVD (singular value decomposition) in order to resolve the minimal collection of eigenvectors that still accurately represents the data. These vectors are used further to identify the unbalanced processes operating in the system. For more details see Remade, F., Kravchenko-Balasha, N., Levitzki, A. & Levine, R. D. Information-theoretic analysis of phenotype changes in early stages of carcinogenesis. Proc. Natl. Acad. Sci. U.S.A 107, 10324-10329 (2010) incorporated herein in its entirety by reference.

Signs of G_{iα} and λ_{α}(k): The term G_{iα} represents the degree of participation of the molecule i in the unbalanced process α, and its sign indicates the correlation or anti-correlation between molecules in the same process. For example, in a certain process α, proteins can be assigned the values: G_{protein1,α}=−0.5, G_{protein2,α}=0.24, and G_{protein3,α}=0, indicating that in this process proteins 1 and 2 are anti-correlated (i.e. protein 1 is upregulated and protein2 is downregulated, or vice versa due to the process α), while protein3 does not participate in the process α. Note that each molecule can take part in a number of unbalanced processes.

Importantly, not all samples are influenced by all processes. The term λ_{α}(k) represents the weight of influence of the unbalanced process α on the sample k. Its sign indicates the correlation or anti-correlation between the same processes in different samples. For example, if the process α is assigned the values: λ_{α}(sample1)=3.1, λ_{α}(sample2)=0, and λ_{α}(sample3)=2.5, it means that this process influences samples 1 and 3 in the same direction, while it does not influence sample 2.

Note that the sign of λ_{α}(k) and G_{iα} represents only correlation or anti-correlation between processes and proteins, respectively. To find the actual change in expression level for each protein i in the tumor k, the term

$${\sum }_{α} {G}_{i α} {λ}_{α} ( k )$$

should be calculated.

As explained above, the zeroth term, ln Xᵢ⁰(k), is the expression level at the balanced state of the system (the most significant process), or the secular invariant term, which was found not to change between patients or in time. This term is utilized as a reference against which the deviation terms are identified. The proteomics dataset contained only regulatory proteins, which are all affected by oncogenic processes, and therefore the most significant process we found is not the balanced state of the system (therefore we designated it as λ₁(k) rather than λ₀(k)). The real balanced state of the system contains proteins and transcripts that are generally very robust and display constant levels in cancer patients as well as in healthy individuals^{16,37}.

In the proteomic dataset, the expression levels of the proteins were normalized according to the median values. Thus, the zeroth term becomes a vector of zeros for all proteins i in all samples, and the dataset is fitted to the unbalanced processes. Equation [1] is reduced to the form:

$$\begin{matrix}ln {X}_{i} ( k ) - ln M = ( ln {X}_{i}^{0} ( k ) - ln M ) - {\sum }_{α = 1} {G}_{i α} {λ}_{α} ( k ) & [ 2 ] \\ ln \frac{{X}_{i} ( k )}{M} \approx - {\sum }_{α = 1} {G}_{i α} {λ}_{α} ( k ) & \end{matrix}$$

where M represents the median value.

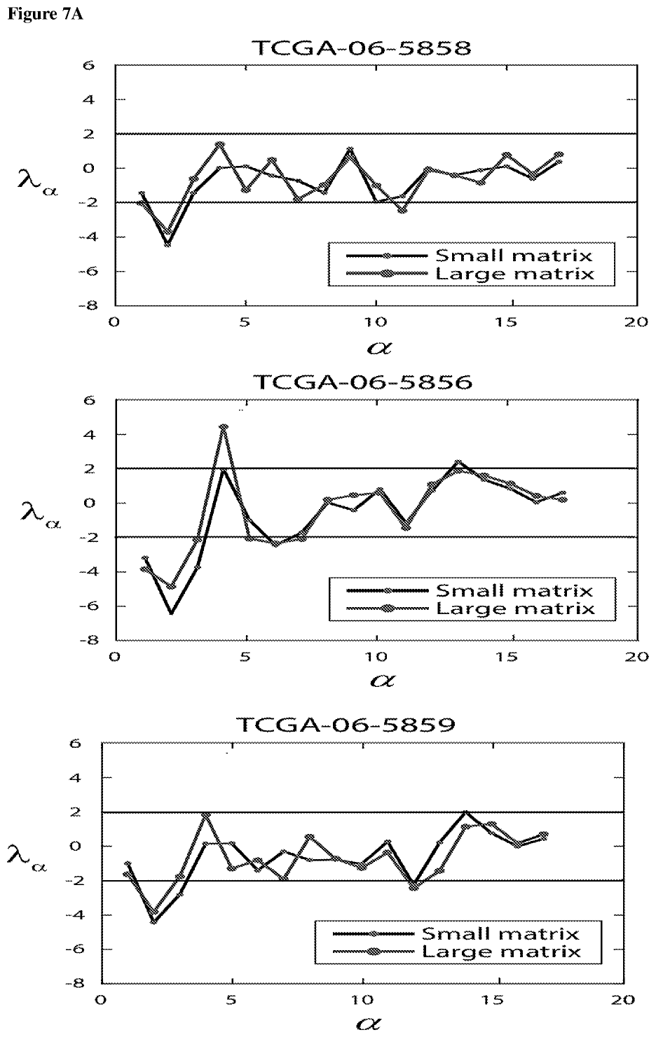

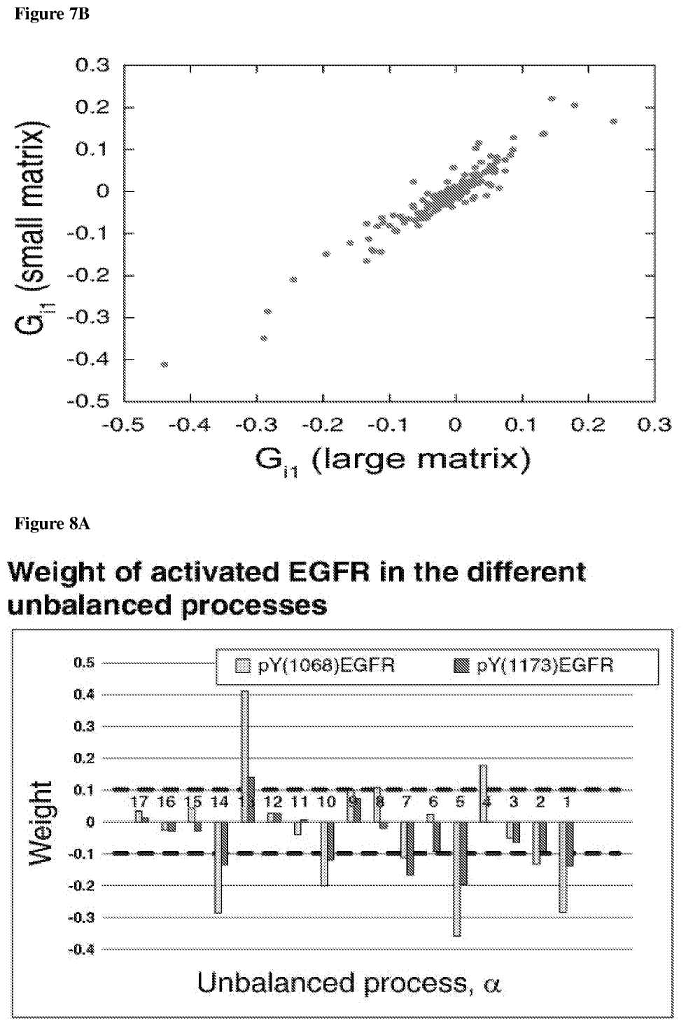

Theoretically, the number of constraints that operate on the system is limited by the smaller dimension of the input matrix. In our case it is limited by the number of macromolecules measured, i.e. in the current dataset, the levels of 181 proteins were measured, and therefore a maximum of 181 constraints, or unbalanced processes that operate on the system could have been found. However, most of these processes are insignificant, i.e. have a negligible weight, 4, in all samples. Our analysis revealed only 17 significant unbalanced processes, which reproduce the experimental expression levels of the proteins in the dataset. FIGS. 7A-B demonstrates the robustness of surprisal analysis and shows that the size of the dataset studied here is large enough. A smaller dataset, of 100 tumors of each type, did not change the results of our analysis.

Integration of different datasets. The following transcriptomic datasets have been obtained from GEO database: GSE17920 (Lymphoma), GSE31684 (Bladder cancer), GSE54129 (Gastric cancer), GSE71222 (Colorectal cancer), GSE82173 (Breast cancer). After deleting the low expression genes on each of the datasets we have 20708 common transcripts for all the datasets. In the section “The 12 unbalanced processes identified are active in other cancer patients” the additional dataset of pancreatic cancer (GSE15471) was added. The list was merged with the previous datasets and the same list of genes were taken.

In every analysis we used singular value decomposition on each analyzed dataset organized as a matrix Y of the logarithms of the expression levels. Each column corresponds to entries for a particular patient, indexed by k. Each row are the entries for a particular transcript indexed by i. The rectangular data matrix Y is used to form two square matrices, Y^{T}Y and YY^{T} where T denotes the transpose. These two matrices have the same non-zero and positive eigenvalues. The largest common eigenvalue is denoted ω₀². For the raw data this eigenvalue depends on the dataset. The regularization we do is that we shift the value of this eigenvalue such that it has a common value for different data sets. We could also shift the (common) trace of the square matrices to have the same value for different data sets. Our compelling reason for not doing that is that by shifting only the value of ω₀² we keep all the other eigenvalues and eigen functions unchanged. This is cardinal because these determine the deviations from the balanced state. Our regularization is insured to shift only the overall intensity of the balanced state.

We first prove that our regularization scales only the overall intensity of the balanced state. Consider the expansion of the matrix Y^{T}Y in terms of its eigenvalues and eigen functions, in decreasing order of the eigenvalues so that ω₀² is the largest

Y^{T}Y=Σ_{α=0}V_{α}ω_{α}²V_{α}^{T} [3]

Here V_{α} is an eigenvector of Y^{T}Y with the eigenvalue ω_{α}². The different eigenvectors of the square matrix are orthogonal. So, if we only rescale ω₀² we get a new matrix Y^{T}Y but with all the other eigenvalues and eigenvectors guaranteed unchanged. It is this new matrix that we take as our regularized data.

How do we determine the change in the eigenvalue ω₀²? We take one data set as a reference. We used colorectal cancer because its eigenvalue ω₀² was the largest. We added a constant to the eigenvalues of other datasets such that the new, shifted, value (ω₀+δω₀)², is the same as the eigenvalue of colorectal cancer.

The procedure described above leaves the deviations from the stable state unchanged. What does it do to the stable state? In our approach (4, 14) the stable state for transcript i in patient k is denoted as Xᵢ⁰(k), see equation [1]. We write Xᵢ⁰(k) as ln Xᵢ⁰(k)=−Gᵢ₀λ₀(k). As discussed in the main text, λ₀(k) is the scale of the balanced state in patient k. We expect that in the stable state all patients have the same level of transcript i. But unavoidable noise means that this is almost but not exactly correct. The exact result is that λ₀(k)=ω₀V₀(k). Here V₀(k) is the k'th element of the eigen vector V₀. We do NOT impose the condition that the values V₀(k) are all equal. Instead we use the fluctuations in the values of the V₀(k)'s as fitted to the data as an estimate of the noise. For the datasets we use these are typically below 6%. Shifting the value of ω₀ shifts the value of λ₀(k) and hence shifts the value of ln Xᵢ⁰(k) as determined from the raw data set to a new value

ln Xᵢ⁰(k)=−Gᵢ₀(λ₀(k)+δλ₀(k)) [4]

This is the value of the stable state that we will use.

To make the shifting discussion very explicit, consider equation [1] of the main text applied to a particular data set. It will look like:

$$\underbrace{ln {X}_{i}^{d} ( k )}_{{measured intensity}_{{of transcript i}_{{of patient k}_{in datase d}}}} = \underbrace{ln {X}_{i}^{od} ( k )}_{{intensity of transcript i}_{{in the balanced state}_{{of patient k}_{in dataset d}}}} - \underbrace{{\sum }_{α = 1} {G}_{i α} {λ}_{α} ( k )}_{{deviations due to the}_{constraints α = 1 , 2 , …}} \qquad [ 5 ]$$

Now subtract the term Gᵢ₀λ₀(k) from both sides. This makes for the equation that describes the expression level of each transcript in the common data set

$$\underbrace{ln {X}_{i} ( k )}_{{measured intensity}_{{of transcript i}_{{of patient k in the}_{common dataset}}}} = \underbrace{ln {X}_{i}^{o} ( k )}_{{intensity of transcript i}_{{in the balanced state}_{{of patient k in the}_{common dataset}}}} - \underbrace{{\sum }_{α = 1} {G}_{i α} {λ}_{α} ( k )}_{{deviations due to the}_{constraints α = 1 , 2 , …}} \qquad [ 6 ]$$

Equation [6] is just equation [1]. The dependence on the particular data set is eliminated by shifting the balanced state. This procedure allows gathering all the 527 samples of five different data sets in the same matrix for carrying out the surprisal analysis.

Calculation of threshold values. To calculate threshold limits for λ_{α}(k) values the standard deviations of the levels of the stable proteins in this dataset were calculated (e.g. those that did not exhibit significant variations between the different patients). Those fluctuations were considered as baseline fluctuations in the population of the patients which are not influenced by the unbalanced processes. Using standard deviation values of these proteins the threshold limits were calculated as described previously⁴¹.

To find which unbalanced process are important in every patient we calculate the error bars for the each sample and an error limit for each type of cancer. Briefly the error bars calculated for each sample are a strict upper bound on the error of the λ_{α} expressed in terms of the error measures and a covariance matrix, see equation [9], whose elements are indexed by patients.

δλ_{α}(k)≤sΣ_{β}(M⁻¹)_{αβ}(M_{ββ})^{T} [7]

s is the patient dependent fold error summed over all expression levels:

s(k)²=Σᵢ(δ Ln Xᵢ(k))²Xᵢ(k) [8]

Here δ Ln Xᵢ(k)=δXᵢ(k)/Xᵢ(k) is the fold error in the expression level of transcript i. As a simple example, it equals 0.1 to represent an experimental error of 10%.

The elements M_{αβ} are the elements of the covariance matrix and are patient dependent because the expression levels vary with different patient.

M_{αβ}=<G_{α}G_{β}>=ΣᵢG_{iα}G_{iβ}Xᵢ(k) [9]

The upper bound given by equation [7] is a strict upper bound and is the patient dependent result when careful analysis is required.

To generate threshold bounds we calculated the standard deviations of the levels of the stable transcripts. The average standard deviations of around 200 transcripts having the lowest standard deviation values were taken. Those fluctuations were considered as baseline fluctuations in the population of the patients which are not influenced by the unbalanced processes. Using standard deviation values of these transcripts the threshold limits were calculated as described previously by us (N. Kravchenko-Balasha, J. Wang, F. Remacle, R. D. Levine, J. R. Heath, Glioblastoma cellular architectures are predicted through the characterization of two-cell interactions. Proc Natl Acad Sci USA. 111, 6521-6526 (2014) herein incorporated by reference. In the transcriptomics dataset—the average standard deviations of ln(Xᵢ(k)) calculated from around 200 stable transcripts of lymphoma was 0.1455 leading to an upper and lower bound of +/−21, in bladder cancer the average standard deviation was 0.132 leading to an upper and lower bounds of +/−19, in gastric cancer the average standard deviation was 0.125 leading to an upper and lower bounds of +/−18, in colorectal cancer the average standard deviation was 0.1456 leading to an upper and lower bounds of +/−21, in breast cancer the average standard deviation was 0.087 leading to an upper and lower bounds of +/−12.5 and in pancreatic cancer the average standard deviation was 0.902 leading to leading to an upper and lower bounds of +/−13. Only those samples having 4(k) values above the threshold limit and error bars above 0, were considered to be influenced by that particular unbalanced process.

We find that only 12 unbalanced processes had λ_{α}(k) values that exceeded the noise.

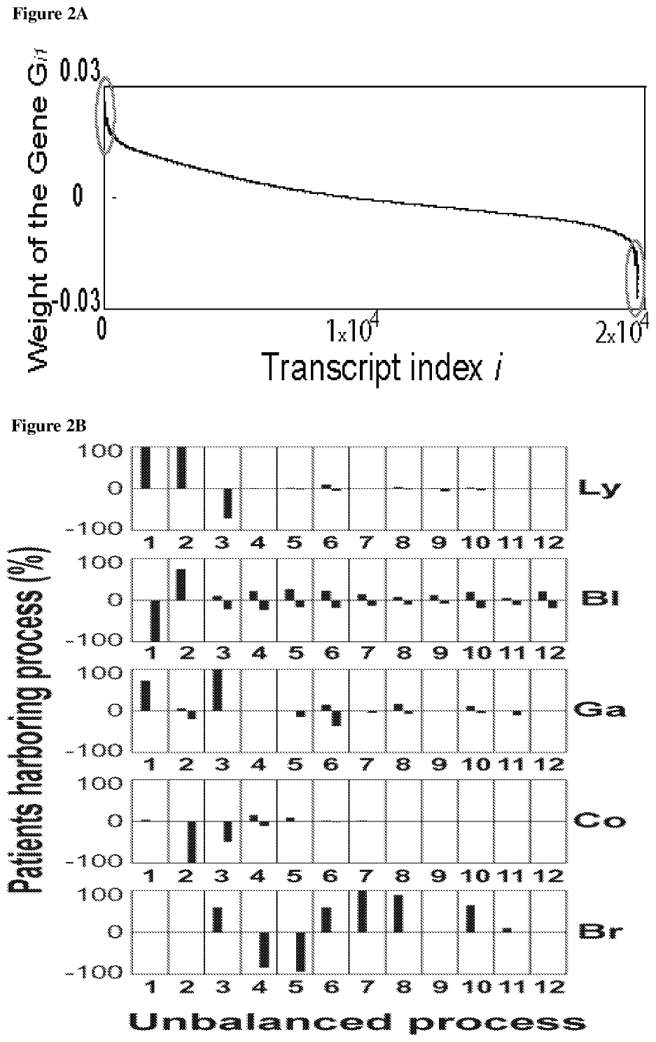

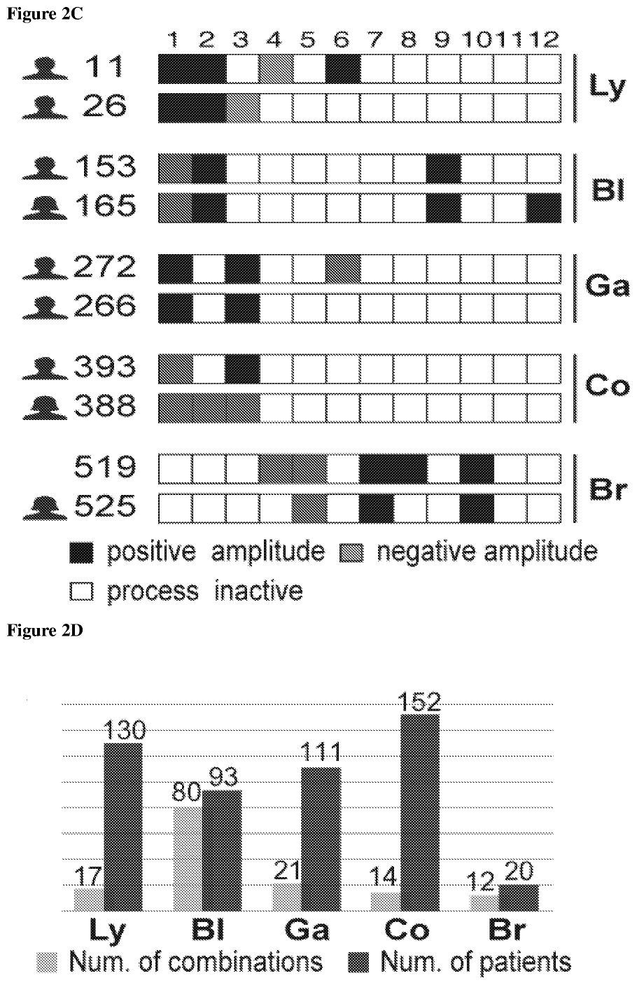

Calculation of patient specific combinations of unbalanced processes. The combinations presented in Table 2, were generated using λ_{α}(k)(α=1,2,3, . . . ) values that exceeded threshold limits and had error bars above 0. For example for a patient k in bladder cancer (transcriptomics dataset), if λ_{α}(k)>19 or λ_{α}(k)<−19 (and is therefore significant according to calculation of threshold values) and its error bars above 0 then process α considered as significant in the patient k. In this way for each patient, the significant unbalanced processes were identified, and the combinations of processes generated for each patient specifically. Table 2 lists combinations of processes for some patients. 144 unique combinations that were identified. FIG. 2C shows their frequency in each cancer type and their overall frequency in all 506 tumors.

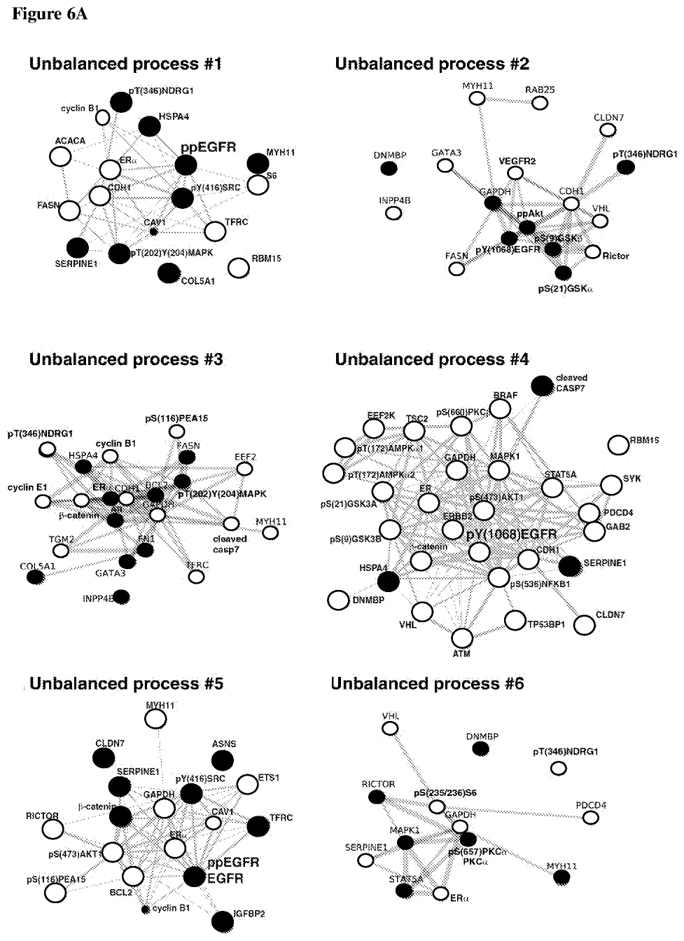

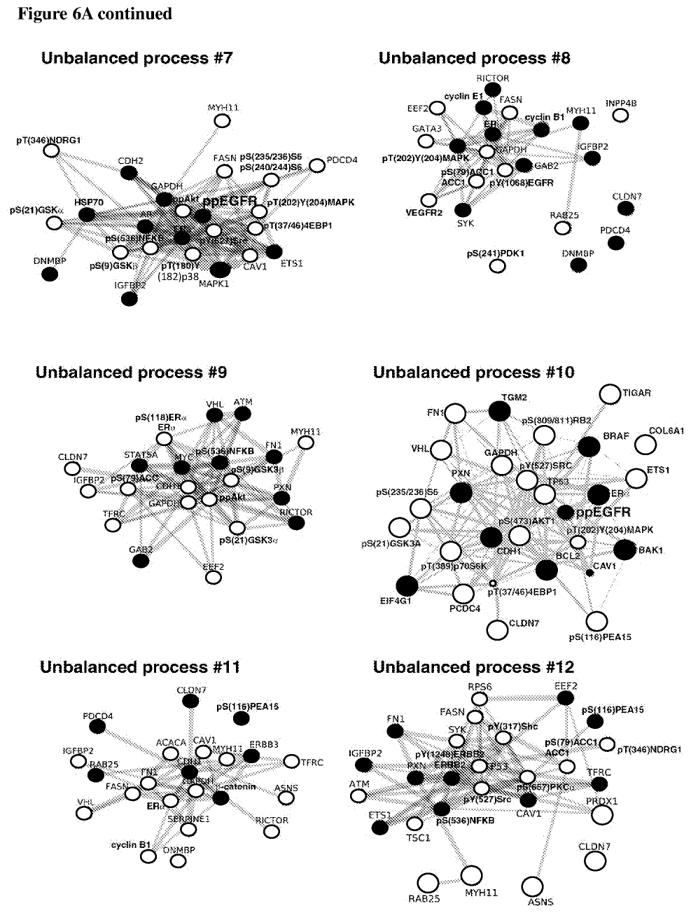

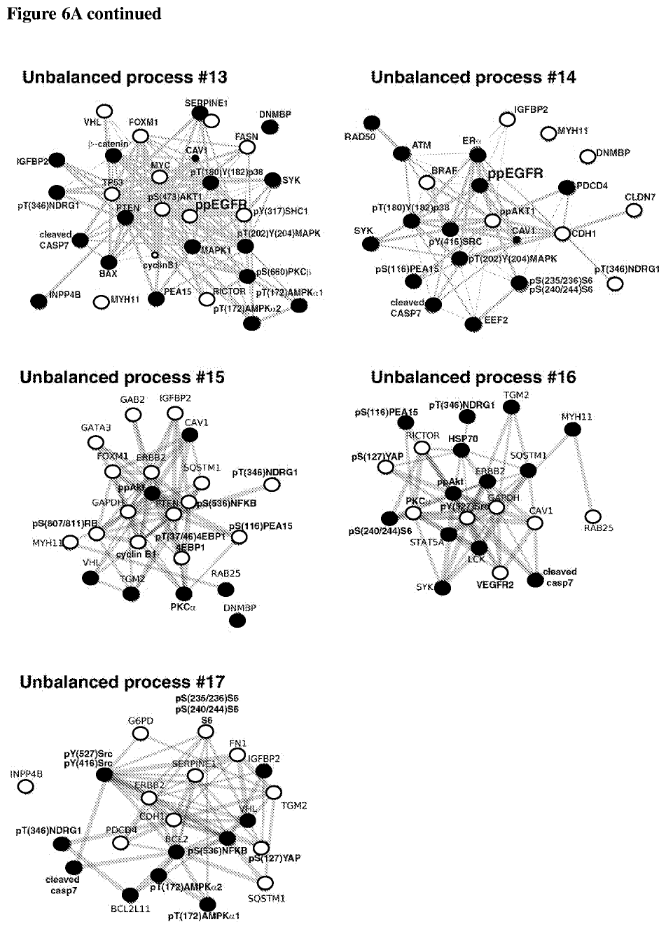

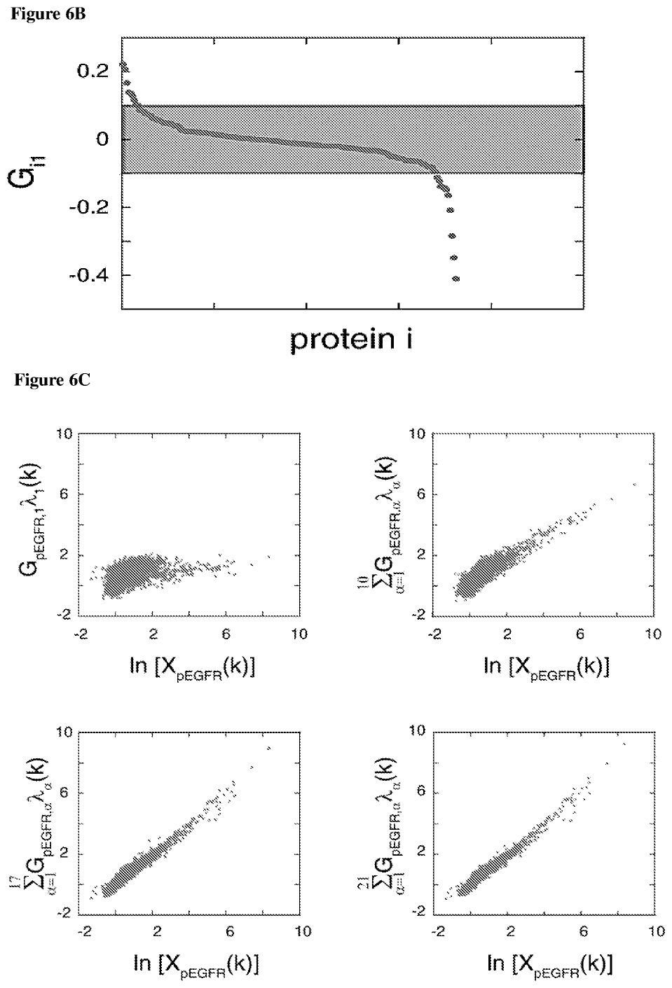

Generation of functional networks. The functional networks presented in FIG. 6A were generated using a python script (written with the assistance of Mr. Jonathan Abramson). The goal was to generate a functional network according to STRING database, where proteins with negative G values are marked white and proteins with positive G values are marked black, in order to easily identify the correlations and anti-correlations between the proteins in the network. The script takes as an input the names of the genes in the network and their G values, obtains the functional connections and their weights from STRING database (string-db.org), and then plots the functional network (using matplotlib library).

Note: Since the antibodies against pY(1248)ErbB2 and pY(1068)EGFR were noticed to cross react in the original RPPA assay, the following corrections were made to our analyses: In unbalanced processes in which both pY(1248)ErbB2 and pY(1068)EGFR were significant, EGFR was considered to be active only if pY(1173)EGFR was significant as well. pY(1248)ErbB2 was considered to be a false-positive result and was thus omitted from these processes. Therefore, pY(1248)ErbB2 was omitted from the unbalanced processes α=1, 5, 10, 13, 14.

Calculation of barcodes. The barcodes presented herein were generated using a python script. For each patient, λ*_{α}(k) (α=1, 2, 3, . . . , 17) values were calculated as follows: If λ_{α}(k)>2 (and is therefore significant according to calculation of threshold values) then λ*_{α}(k)=1, if λ_{α}(k)<−2 (significant according to threshold values as well) then λ*_{α}(k)=−1, and if −2<λ_{α}(k)<2 then λ*_{α}(k)=0. Table 2 contains examples of unique barcodes that were identified. The results are shown graphically in FIG. 2A.

### Example 1: Using Information Theory to Identify Patient-Specific Ongoing Cancer Processes

Tumors are biological systems in which the balanced homeostatic state has been disturbed due to genomic and environmental factors, or constraints. These constraints bring about an imbalance in the tissue and result in abnormal gene expression levels reflecting ongoing unbalanced molecular processes. To quantify the imbalance, we utilize the thermodynamically motivated information-theoretic surprisal analysis.

Surprisal analysis identifies which gene products are at their balanced state level for every single tumor. We have previously shown that this balanced state is robust and remains unchanged between normal and cancer tissue and even between different organisms (Kravchenko-Balasha N, et al., 2016, A Thermodynamic-Based Interpretation of Protein Expression Heterogeneity in Different Glioblastoma Multiforme Tumors Identifies Tumor-Specific Unbalanced Processes. J Phys Chem B. doi:10.1021/acs.jpcb.6b01692). The analysis further uncovers the complete set of constraints that operate in the system, including the genes that are affected by these constraints and have thus deviated from their balanced state levels.

The equation used herein represents the logarithm of the experimental transcript expression level, ln Xᵢ(k), of a measured transcript i, in every patient k as:

$$\underbrace{ln {X}_{i} ( k )}_{{logarithm of}_{{measured intensity}_{{of transcript i}_{in patient k}}}} = \underbrace{ln {X}_{i}^{o} ( k )}_{{logarithm of}_{{intensity of transcript i}_{{in the balanced state}_{of patient k}}}} - \underbrace{{\sum }_{α = 1} {G}_{i α} {λ}_{α} ( k )}_{{logarithm of deviations in the}_{{intensity of transcript i due}_{{to the constraints α = 1 , 2 , …}_{as a sum over these processes}}}} \qquad [ 10 ]$$

where in Xᵢ⁰(k) is the logarithm of the expression level of the transcript i at the balanced state, and the sum, Σ_{α=1}G_{iα}λ_{α}(k), represents the deviations in the logarithm of the expression level of this transcript from the balanced state level due to the environmental/genetic constraints that operate in the system.

The balanced state term can be represented as ln Xᵢ⁰(k)=−Gᵢ₀λ₀(k) (7), allowing to calculate an amplitude for the balanced state, λ₀(k), for every tumor k and the extent of the participation of each individual transcript i, Gᵢ₀, in the balanced state process, α=0. The experimental data we wished to analyze in this study originated from a number of different datasets. As mentioned above, we expect that the expression level of transcript i in the balanced state, Xᵢ⁰(k), would be common to all patients and not depend on the patient index, k.

The unbalanced processes are indexed by α=1, 2, 3. Each constraint significantly influences only a subset of transcripts in a similar way by causing the collective deviations of the transcript levels (up or down) from the balanced level. Therefore, a constraint represents an unbalanced process in the system. Each unbalanced process can consist of several biological pathways. For example, proteins involved in aerobic glycolysis and MAPK (Mitogen-activated protein kinases) signaling pathways can deviate in a coordinated manner from the balanced state and thus participate in the same unbalanced process.

Several unbalanced processes may operate in each tumor, and each transcript can participate in several unbalanced processes due to the non-linearity of biological networks.

Singular Value Decomposition, SVD, is used as a mathematical tool to determine the two sets of parameters required in surprisal analysis to represent the unbalanced processes: (a) The λ_{α}(k) values, denoting the amplitude of each constraint (unbalanced process), in every tumor k; (b) The Gᵢₐ values, denoting the extent of the participation of each individual transcript i in the specific unbalanced process, α (7). Note that the weight, Gᵢₐ, of transcript i is the same for all patients (i.e. is independent of k). Hence, the structure of every process α remains constant. The amplitude, λₐ(k), determines whether process α is active in the patient k, and to what extent.

Our goal was to utilize surprisal analysis to classify tumors according to the tumor-specific sets of constraints that deviate the cancer tissues from the stable, balanced state. We suggest that such a classification is essential to improve personalized cancer diagnostics.

### Example 2: Integrating Biological Datasets to Study Inter-Patient Heterogeneity (Transcriptomics Dataset)

The field of personalized medicine has been accelerating and a massive amount of gene expression data regarding different types of cancer is becoming available. 5 different datasets containing transcriptomic data were selected for analysis, each comprising samples from a different type of cancer: lymphoma, bladder cancer, gastric cancer, colorectal cancer and breast cancer. The gastric cancer dataset included 21 normal samples as well. The total number of samples was 527. A concurrent analysis of different datasets will allow identification of the altered biological processes that characterize the inter-patient heterogeneity. Additionally, a large-scale analysis should uncover the patient-specific sets of unbalanced processes with better signal to noise.

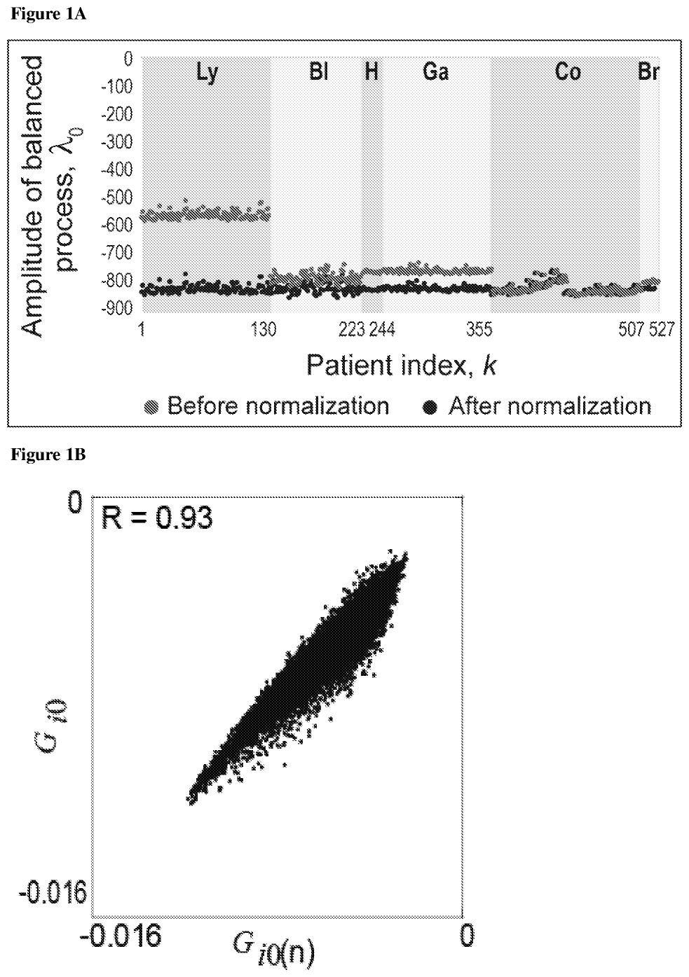

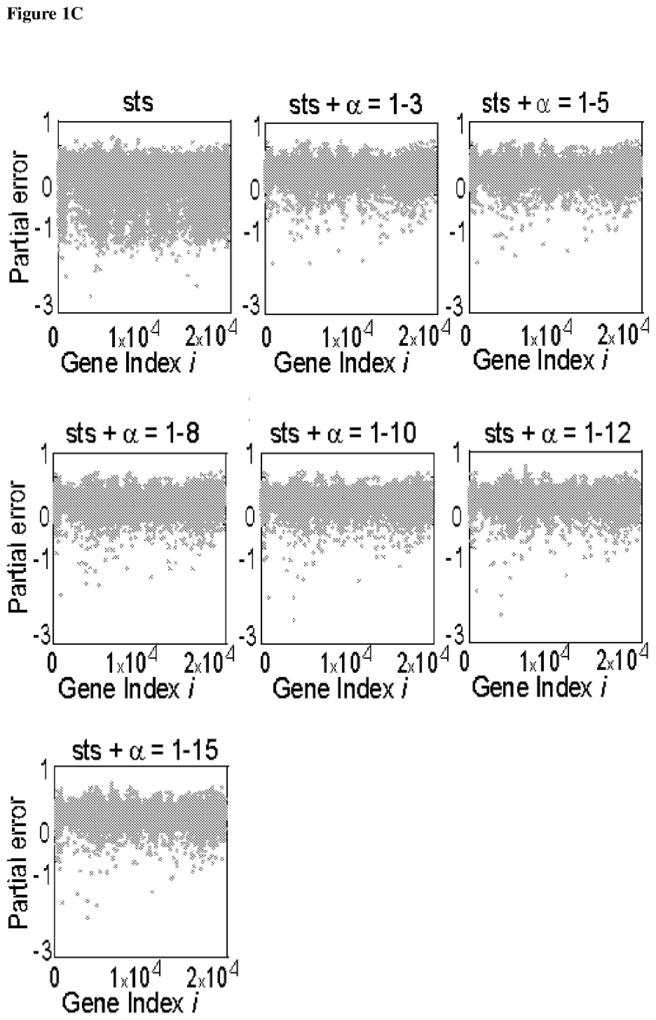

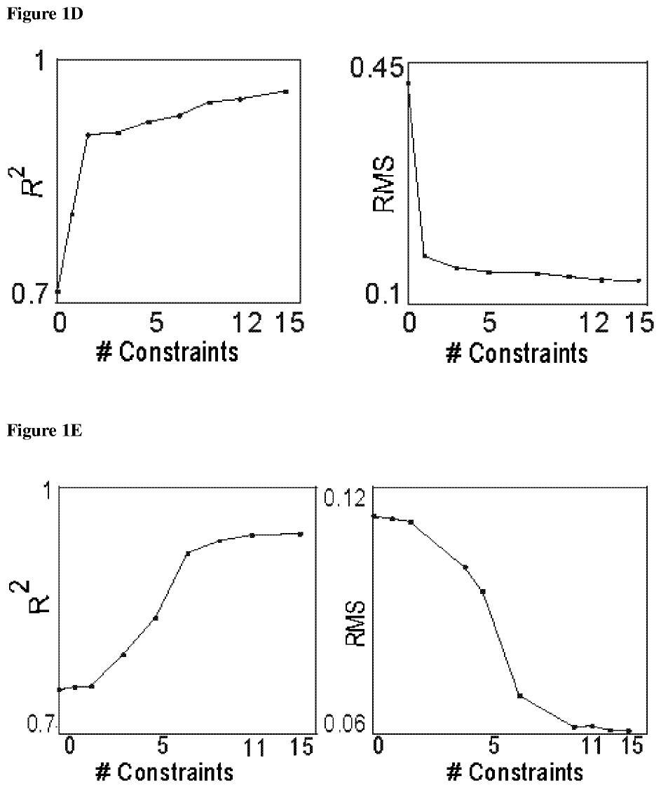

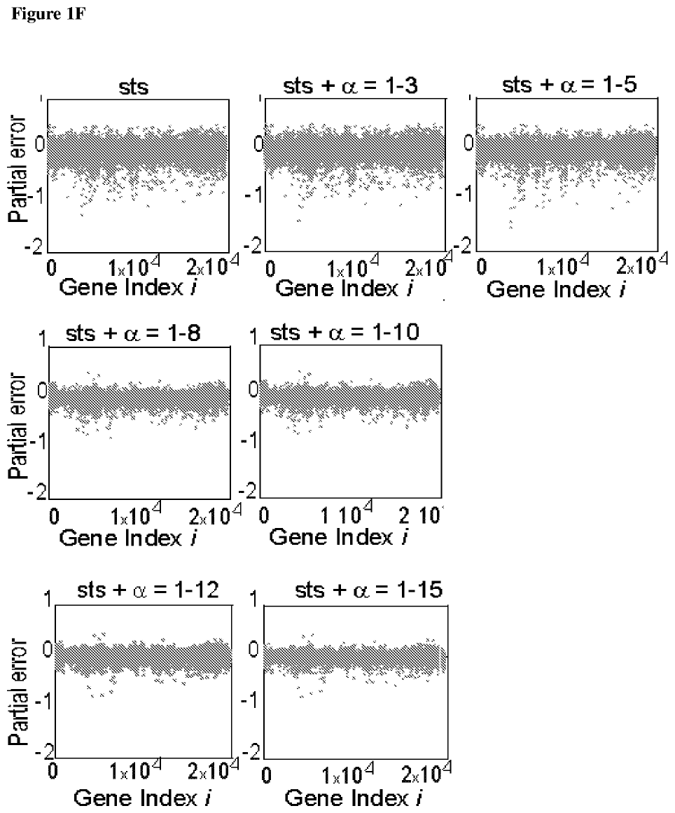

As expected, surprisal analysis of the 5 datasets identified a common balanced state for each type of cancer, represented by an invariant amplitude of the balanced state λ₀(k) for all patients, k, of the specific cancer type, including the normal gastric samples (FIG. 1A). This result corresponds to our previous findings demonstrating the robustness of the balanced process. The levels of more than 700 transcript probes out of ˜20,000 probes were well-fitted by the balanced term alone and were not influenced by any unbalanced process. These transcripts participate in the homeostatic functions of the cell, such as protein and RNA metabolism, and the cell cycle.

Following determination of the balanced state term separately for each dataset, the intensities of the different sets were normalized and converted to a common scale, such that all 5 datasets shared a common balanced state term (FIG. 1A). Importantly, the transcript composition of the steady state remained invariant before and after the normalization, suggesting that the intensity differences reflected experimental artifacts and not biological differences (FIG. 1B). The thermodynamic-based approach underlying surprisal analysis is what enables to do such a normalization. Importantly, we demonstrate that this normalization does not influence the amplitudes of the unbalanced processes nor the weight of individual transcripts in these processes (see Methods).

The notion that the balanced state is common to normal and cancerous tissues is highly significant, because it suggests that the search for the tumor gene markers should focus only on the unbalanced processes, greatly reducing the number of possible targets.

### Example 3: The Inter-Patient Heterogeneity Among 506 Patients is Characterized by 12 Unbalanced Processes (Transcriptomics Dataset)

Our next step was to inspect the unbalanced processes that characterized the 506 tumors (527 total samples not including the 21 normal gastric samples). The analysis revealed that 12 unbalanced processes significantly sufficed to reproduce the deviations from the balanced state across the 506 tumors of 5 types. The amplitudes of the processes and how they are selected based on the analysis of the experimental errors (or fluctuation) in the transcripts expression levels are given herein above in the Methods section. We used three different methods to identify the processes in light of experimental inter-patient variability: (i) Error limits were based on the expression levels on the basis of the most stable transcripts, (ii) Error bars for each patient were computed and (iii) Convergence of the deviations from the balanced state, equation [1], to the measured data.

Typically, each patient is characterized by a subset of about 5 or fewer processes as determined by the three methods discussed above. Details for calculation of exact number of unbalanced processes can be found in the Methods section. Further, to find the exact number of the unbalanced processes we calculate error bars and threshold limits as described herein below. To check that the number of unbalanced processes is meaningful we calculate for every patient how many processes are needed in order to fully reproduce his experimental data. This is shown for one exemplary patient 193 in FIGS. 1C-D and for exemplary patient 510 in FIGS. 1E-1F. The partial error, representing variance in expression data, was calculated, using the formula (Ln Xi(k)−Σ_{α=1}G_{iα}λ_{α}(k))/LnXi(k). We expect to achieve the reduction in a partial error as we add more constraints. Indeed, as shown in FIG. 1C the errors remain essentially the same, despite adding the additional constraints (FIGS. 1C and 1F, plots six and seven of each), which means that the variance in the expression levels is due to noise and not due to unbalanced processes. The R² was calculated by plotting the natural logarithm of the experimental data (LnXi) vs ΣG_{iα} λ_{α} (k) for α=0, 1, 2, 3, 5, 8, 10, 12, 15. As we add more constraints, we expect to get higher correlation between the experimental data and the theoretical calculation, reaching the highest level of correlation when the experimental data is fully reproduced by the theoretical fitting (FIGS. 1D and 1E, left panels). The plot reaches a plateau after the 12^{th} constraint. This indicates that after the 12^{th} constraint the data includes mainly noise. Root Mean Square value is calculated to demonstrate the decrease in the variability when 12 processes are included in the calculation: The Root Mean Square (RMS) values were calculated for the partial error of each α=0, 1, 3, 5, 8, 10, 12, 15 and plotted (FIGS. 1D and 1E, right panels). The variation, which was high when only α=0 was included, becomes very low when calculated for α=0-12, and reaching a plateau afterwards, again showing that 12 constraints are enough to fully reproduce the entire dataset.

To assign a biological meaning for each constraint, transcripts with the most significant Gᵢₐ values (FIG. 2A) were classified into biological categories according to Gene Ontology (GO) database. Those transcripts which have the highest positive values (>0.0125, located on the positive tail) are highly expressed if the λ₁ value is positive in a patient. These genes have low expression levels in patients with a negative λ₁ value. Similarly, the genes with most negative values (<−0.015, located on the negative tail) are highly expressed in patients with negative λ₁ values and have low expression levels in patients with positive λ₁ values. Similarly, for all other constraints α=2, 3, 4 . . . 12 only the transcripts located on the tails of the sigmoid graphs were further analyzed. For every constraint we find that levels of about 500-1000 RNA transcripts deviate significantly in positive or negative direction from the balanced state. Only those transcripts that were located on the tails were included in the classification of biological categories using David database. Some transcripts are involved in only one constraint, e.g. GRB2, PTK2B, and CALM3, whereas others participate in 2 or more unbalanced processes, such as EGFR, PD-L1 (CD274), CD44, IRS2, EIF4E, and CDK1. Each unbalanced process can include multiple (sometimes overlapping) biological categories. Importantly, we found that in every cancer type, one or more unbalanced processes are shared by all of the patients of this cancer type (FIG. 2B). For example, all of the lymphoma patients were found to harbor the process α=1 with a positive amplitude (λ₁(k)>0), which we define as process 1+(FIG. 2B). Process 2+ was found in all patients of lymphoma as well (FIG. 2B). Genes involved in these processes were classified to multiple categories, for example, B-cell signaling, cell proliferation, platelet deregulation and DNA repair. Recall that the weights, G_{iα}, are independent of the patient index, k, and that it is the amplitude, λ_{α}(k), that determines whether a process is active in the specific patient. The sign of λ_{α}(k) determines the direction to which the process deviates the transcripts. Thus, if all lymphoma patients harbor process 1+, it means that process 1 deviates the transcripts in the process in the same manner in all lymphoma patients, i.e. upregulates or downregulates them. Process 1− was found in all patients of bladder cancer (FIG. 2B), and includes genes involved, for example, in intracellular signal transduction and GTPase activation. Process 3+ appeared in all patients of gastric cancer (FIG. 2B), and includes genes involved in angiogenesis and anti-apoptosis. Process 2− appeared in all patients of colorectal cancer (FIG. 2B), and includes genes involved in IL4 and IL10 production, NFkB signaling. The breast cancer patients were all found to harbor process 7+(FIG. 2B), which includes VEGFR signaling and glucuronidation (a mechanism of intrinsic drug resistance). The finding that certain unbalanced processes are shared by all patients of a particular cancer type is consistent with our earlier findings that there is a dominant process that characterizes a particular type of cancer, as compared to normal samples (4, 6, 7). Note, however, that the same process may also appear in other cancer types, possibly less frequently. For example, process 3− is shared by lymphoma, bladder and colorectal cancers (FIG. 2B). This constraint includes transcripts involved, for example, in PGDFR signaling pathway, mRNA processing and splicing. Process 5− appears in bladder, gastric and breast cancers and comprises transcripts involved in, for example, Wnt signaling, cell-cell adhesion and RNA splicing. Processes of higher index appear in a smaller number of patients.

### Example 4: From Unbalanced Processes to Patient-Specific Signatures (Transcriptomics Dataset)

12 unbalanced processes repeat themselves across the 506 tumors. However, not all processes are active in all tumors. Every individual tumor harbors a specific subset, or signature, of active unbalanced processes (FIG. 2BC-D). Typically, every patient can be accurately represented by a combination of 1-5 ongoing processes (FIG. 2C). Although cancer patients can have the same type of cancer, they may harbor different sets of unbalanced processes. For example, patients 1 and 26 were diagnosed with lymphoma and have process 1 and 2 in common and differ in the rest of the active unbalanced processes (FIG. 2C). Negative/positive amplitude denotes how the patients are correlated with respect to a particular process (see Methods). For example, patient 165 harbors process 1-, while patient 272 harbors process 1+. Therefore, transcripts that participate in process 1 deviate from their balanced level in opposite directions in these patients.

12 unbalanced processes can be assembled into thousands of unique subsets of 1-5 processes. We found varying degrees of inter-tumor heterogeneity in each of the tumor types (FIG. 2D): 17 combinations of processes were found in the population of 130 lymphoma patients; 80 combinations were found in the population of 93 bladder cancer patients; 21 combinations in the population of 111 gastric cancer patients; 14 unique combinations in the population of 152 colorectal cancer patients; and 12 combinations of processes were found in the population of 20 breast cancer patients. Thus, some cancer types possess a high degree of heterogeneity (e.g. bladder), whereas others, such as colorectal cancer, are significantly less heterogeneous (FIG. 2D).

### Example 5: Similar Gene Expression Levels can Result from Different Combinations of Unbalanced Processes (Transcriptomics Dataset)

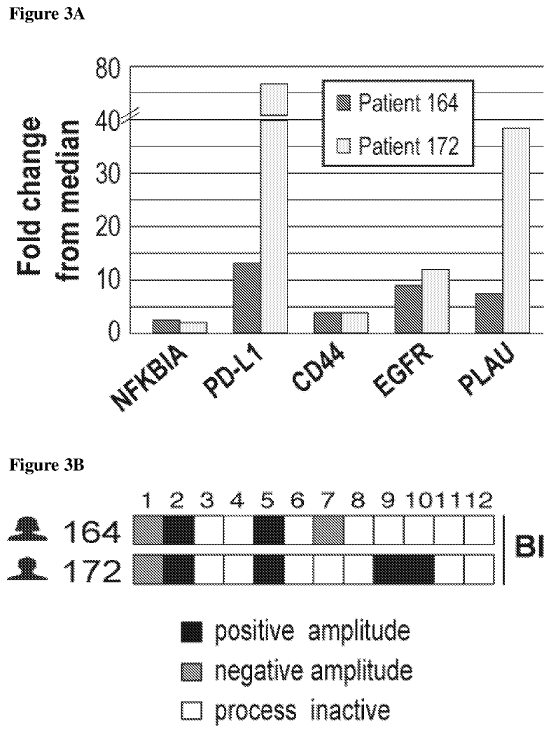

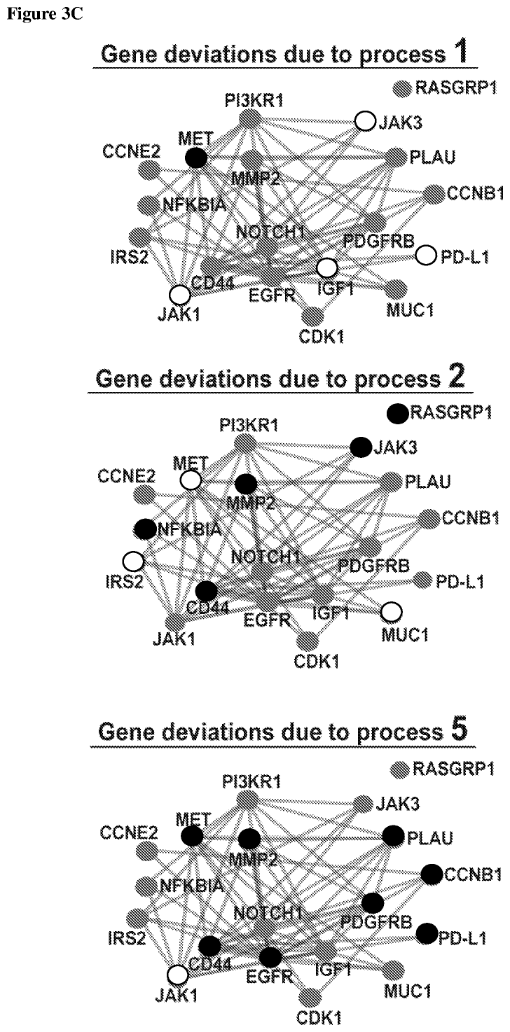

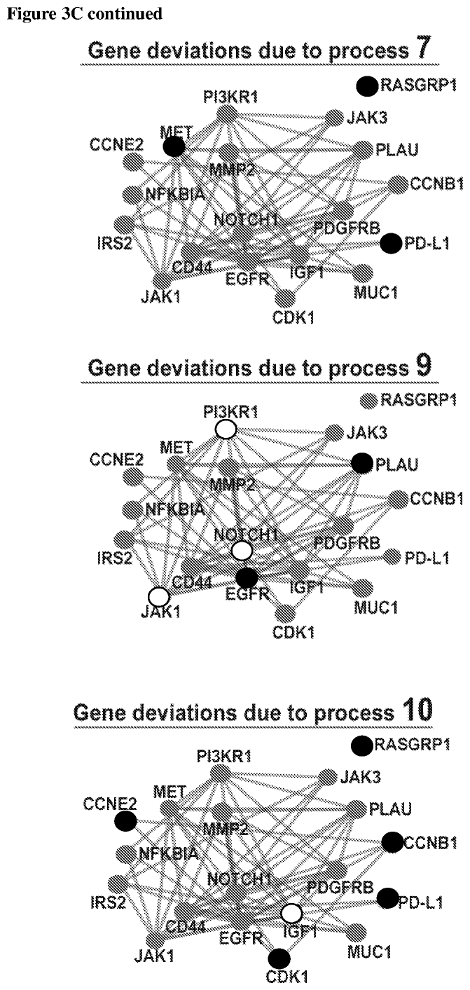

One of the main features of surprisal analysis is its ability to assign transcripts to more than one unbalanced process. For example, epidermal growth factor receptor (EGFR) was found to independently participate in processes 9 and 5; programmed death-ligand 1 (PD-L1, inhibitor of the immune system) participates in processes 5 and 7 (FIG. 3C). Therefore, two patients can display similar gene expression levels, while their tumors may harbor different combinations of unbalanced processes. To demonstrate this point, we selected two bladder cancer patients, indexed 164 and 172, and inspected their tumor-specific experimental expression levels of 5 bladder-cancer associated genes: NFkB, PD-L1, CD44, EGFR, and PLAU (FIG. 3A). In both tumors, these oncogenes were upregulated relative to their median expression level (FIG. 3A). However, surprisal analysis revealed that the tumors are biologically distinct: patient 164 is characterized by a combination of processes 1, 2, 5 and 7, whereas patient 172 harbors a combination of processes 1, 2, 5, 9, and 10 (FIG. 3B). FIG. 3C shows 19 selected genes, and how they were affected by these unbalanced processes in the two patients. In both patients, the induction of NFkB is associated with unbalanced process 2, and the induction of CD44 is associated with processes 2 and 5. However, the induction of other oncogenes was attributed to different processes. For example, in patient 164, PD-L1 induction was attributed to processes 5 and 7, while in patient 172 PD-L1 induction was attributed to processes 5 and 10. Similarly, EGFR was induced by process 5 in patient 164, while in patient 172 it was induced due to processes 5 and 9.

Patients 164 and 172 serve as an example for two patients carrying tumors of the same type, which may present with similar lists of oncogenic biomarkers, even though their tumors are not the same. Classification of tumors according to similar biomarkers, may lead to significant differences between cancer patients in terms of response to treatment, survival prediction, and more. Deciphering the complete altered transcriptional network in every tumor should enable more accurate diagnosis and classification of patients.

### Example 6: The 12 Unbalanced Processes Identified are Active in Other Cancer Patients (Transcriptomics Dataset)

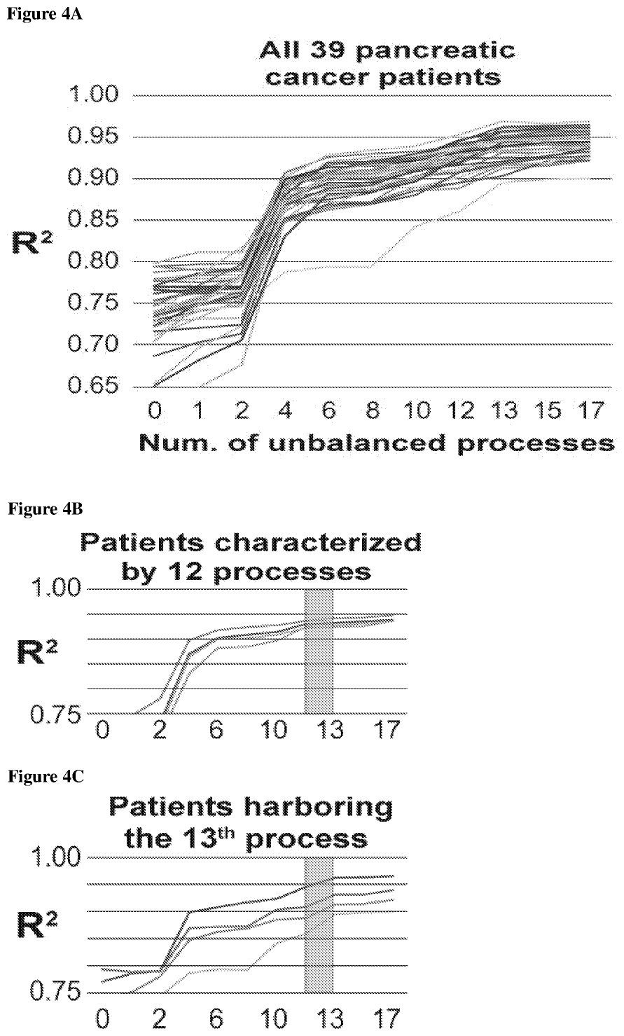

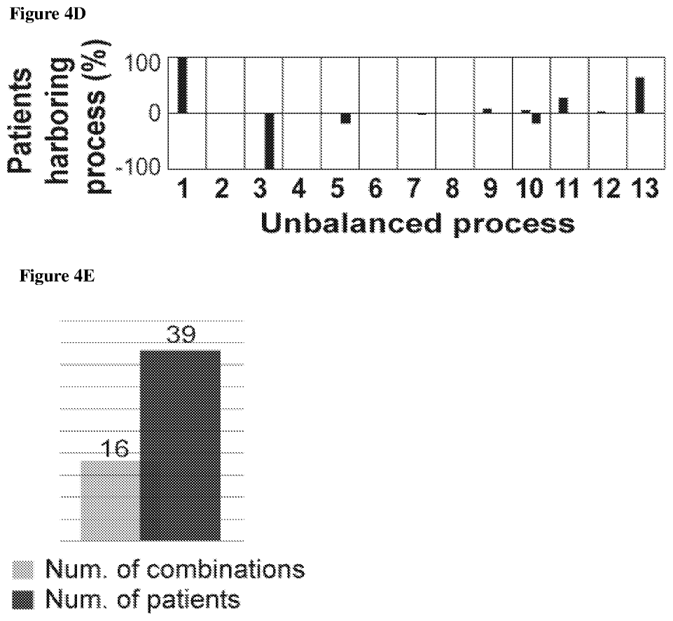

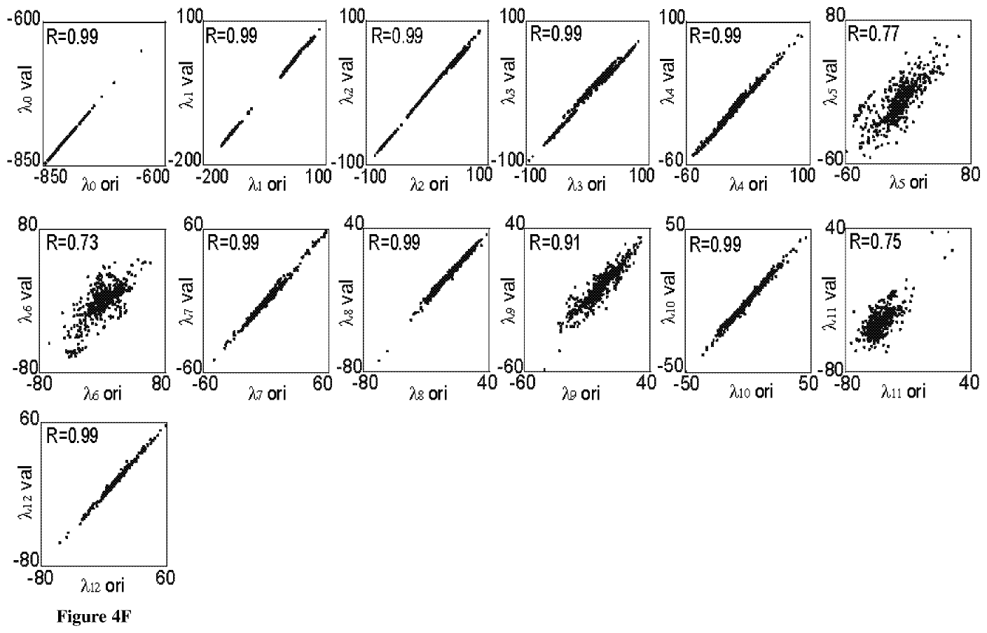

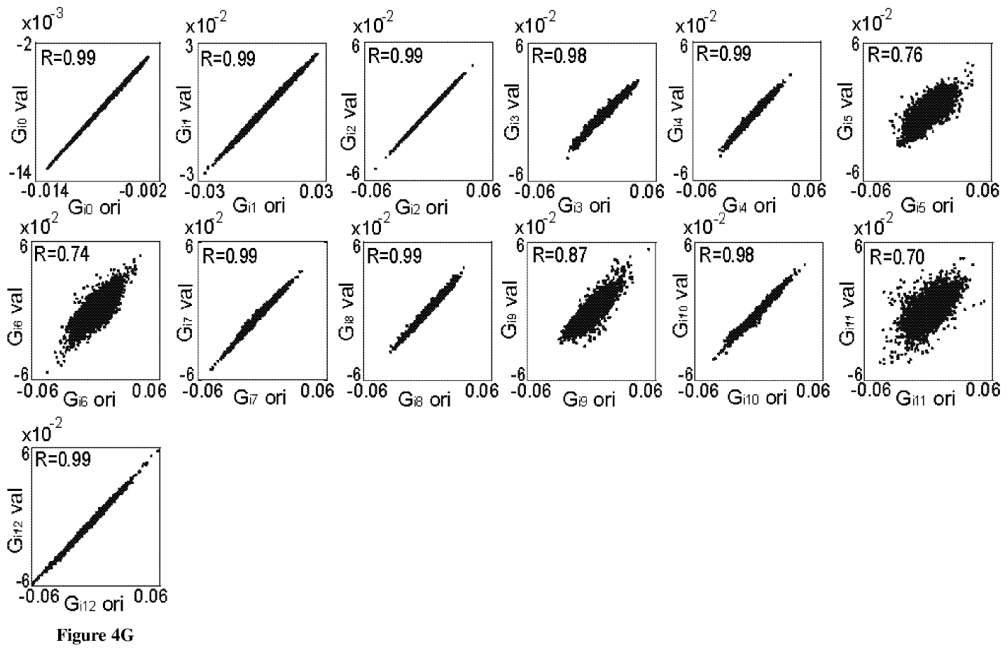

Our next step was to verify whether the 12 unbalanced processes that were identified in the 506 tumors are relevant to other cancer patients as well. To answer this, we obtained an additional dataset, which consists of 39 pancreatic tumors. This additional dataset will be referred to as the validation set. The dataset was merged with the previously analyzed 5 datasets (utilizing the normalization method described hereinabove), and the combined dataset, comprising 566 patients, was analyzed using surprisal analysis (FIG. 4A-E). 13 unbalanced processes were identified in this analysis. Mathematically, 506 unbalanced processes are calculated for each patient. However, not all of them are significant. FIG. 4A shows that all patients reach a plateau after 13 processes, suggesting that the first 13 unbalanced processes are significant, and the rest of the processes represent random noise in the biological system. The amplitudes of the 12 unbalanced processes that were found in the population of the patients before and the first 12 found after addition of pancreatic dataset were compared, attempting to find out whether these are the same processes. The amplitudes of 12 unbalanced processes, which were identified in the first (5 cancer types) dataset, were highly correlated with the amplitudes of the processes after addition of the pancreatic cancer dataset. The correlation coefficient R is above 0.9 for the lambdas 0, 1, 2, 3, 4, 7, 8, 9, 10 and 12 and is above 0.7 for the lambdas 5,6, and 11 (FIG. 4F). Additionally, the weights of the transcripts in every unbalanced process from the first dataset were highly correlated with the weights of the transcripts after addition of the pancreatic cancer dataset. The correlation coefficient R is above 0.9 for the processes 0, 1, 2, 3, 4, 7, 8, 10 and 12 and is above 0.7 for the processes 5, 6, 9, and 11 (FIG. 4G). Thus, strikingly, the first 12 unbalanced processes appeared to be the same 12 unbalanced processes that were identified in the analysis of the original 527 samples and could fully characterized 36% of the pancreatic patients (FIGS. 4B and 4D). The 13^{th} process, which appeared only upon addition of the validation set to the analysis, did not correlate with any of the processes, including higher index processes >12) and was essential for the characterization of the remaining ˜64% of pancreatic patients (FIG. 4C-D). As this process did not appear in the original dataset, it would appear to be a pancreatic cancer-specific constraint. The transcripts involved in unbalanced process 13 were categorized, among others, to the Notch, IL-1, NFkB and EGFR signaling pathways. These pathways are known to be involved in pancreatic cancer.

Interestingly, unbalanced processes 1+ and 3− appeared active in all pancreatic patients (FIG. 4D). Unbalanced process 11, which was found in 14 patients of bladder cancer (FIG. 2A), was active in ˜28% of the validation pancreatic set (FIG. 4D). Process 12, which represented only bladder cancer patients previously (FIG. 2A), was found only in 1 pancreatic patient (FIG. 4D). Overall, 16 different combinations of unbalanced processes were found in 39 pancreatic patients, demonstrating a relatively high inter-patient heterogeneity (FIG. 4E).

### Example 7: Surprisal Analysis for an In-Depth Study of 3467 Tumors of 11 Different Types (Proteomics Dataset)

In order to further broaden the cancers analyzed, we performed surprisal analysis on a proteomic dataset that was obtained by subjecting samples from 3467 TCGA (The Cancer Genome Atlas) solid tumors to reverse phase protein array analysis. The tumors were of 11 different types: Breast (BRCA; n=747), colon adenocarcinoma (COAD; n=334), rectal adenocarcinoma (READ; n=130), kidney renal cell carcinoma (KIRC; n=454), ovarian cancer (OVCA; n=412), endometrial carcinoma (UCEC; n=404), lung adenocarcinoma (LUAD; n=237), head and neck squamous cell carcinoma (HNSC; n=212), lung squamous cell carcinoma (LUSC; n=195), bladder carcinoma (BLCA; n=127), and glioblastoma multiforme (GBM; n=215). The protein array included high-quality antibodies that target 181 proteins and phosphoproteins that play key roles in oncogenesis-related processes, such as proliferation, DNA damage, EMT, invasion, and apoptosis30.

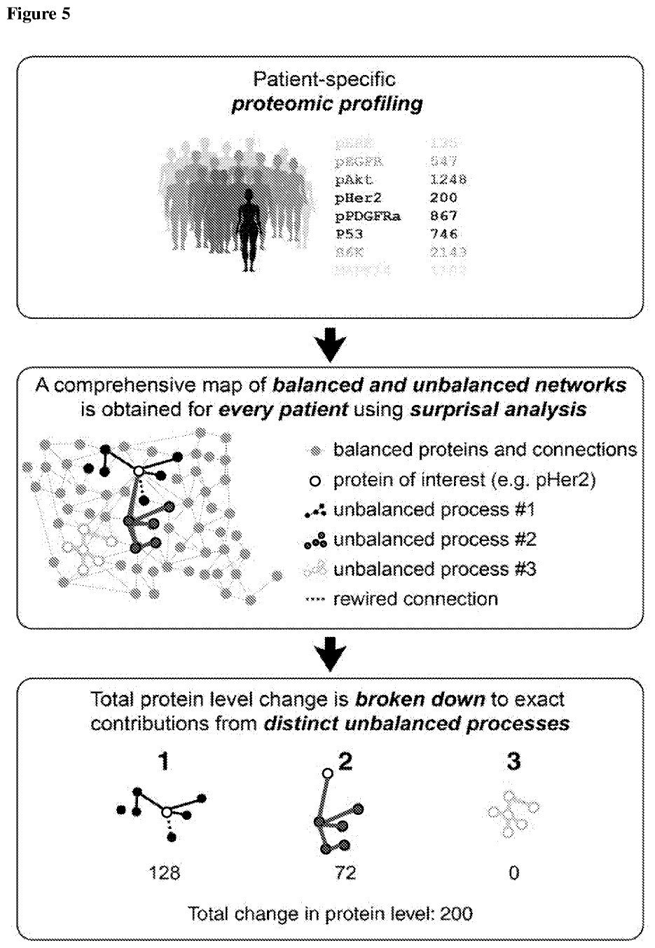

A schematic of the surprisal analysis protocol is provided in FIG. 5. The input for this surprisal analysis is the expression levels of proteins (but can generally be other macromolecule such as genes and transcripts) from multiple patients (FIG. 5, top panel). Based on thermodynamic-like considerations, the proteins are divided into groups in which the expression levels change in a similar, coordinated manner relative to the balanced levels. These groups of proteins represent the unbalanced biological processes that underlie the cancerous phenotypes (FIG. 5, middle panel). Each unbalanced process can comprise a number of signaling pathways that undergo coordinated changes. In the current analysis, the dataset included tumor samples only, and therefore the unbalanced processes identified by surprisal analysis characterize the inter-tumor heterogeneity.

Tumors are complex biological systems that deviate the tissue from the steady state due to various constraints that operate on them. Each tumor can be influenced by different constraints and therefore by a different set of unbalanced processes. For each unbalanced process α, we calculate λ_{α}(k)—the amplitude of this process in every tumor k.

Importantly, due to the non-linearity of signaling networks, each protein can be influenced by a number of different unbalanced processes. For every protein, i, we calculate G_{iα}—the weight of participation of this protein in every unbalanced process α. Hence, for every protein, the total change in expression level can be broken down, such that the contribution of every unbalanced process to the total change in expression level is easily deciphered (FIG. 5, middle and bottom panels). This increases our ability to uncover patient-specific non-linear, as well as rewired, pathways that underlie cancer heterogeneity. Considering that rewired pathways may require different drugs to target them, their identification is crucial.

Refer to Methods for more details regarding the theoretical analysis.

### Example 8: 17 Unbalanced Processes Span the Entire Heterogeneous Unbalanced Signaling Flux in 3467 Tumors (Proteomics Dataset)

We found 17 unbalanced processes in the whole population of 3467 tumors (FIG. 6A). The proteins participating in the different unbalanced processes were assembled into networks, using functional interactions according to STRING database (FIG. 6A). Proteins with positive G_{iα} values are colored black, and proteins with negative G_{iα} values are colored white. Note that the signs of G_{iα} only represents the correlation or anti-correlation between proteins in the process α. To determine the direction of change in protein expression level, the product G_{iα}λ_{α}(k) should be examined (see Methods). The process of determination of thresholds for the amplitudes of the unbalanced processes is described in Methods. FIG. 6B demonstrates how we determined the thresholds for the weights of the proteins in each process. The proteins that take part in the different unbalanced processes were identified as follows: For every unbalanced process α, G_{iα} values were sorted according to their size, and only proteins with significant G_{iα} values were considered to participate in the unbalanced process α. FIG. 6C shows how 17 processes suffice to reproduce the experimental data. To show that the first 17 unbalanced processes are enough to reproduce the dataset, we looked whether the changes in the experimental protein expression levels can be fitted well by 17 unbalanced processes that appeared to be significant (exceed the threshold limits). For example, for every tumor k, the theoretical expression level of pEGFR in this tumor

$$( {\sum }_{α = 1}^{n} {G}_{pEGFR , α} {λ}_{α} ( k ) )$$

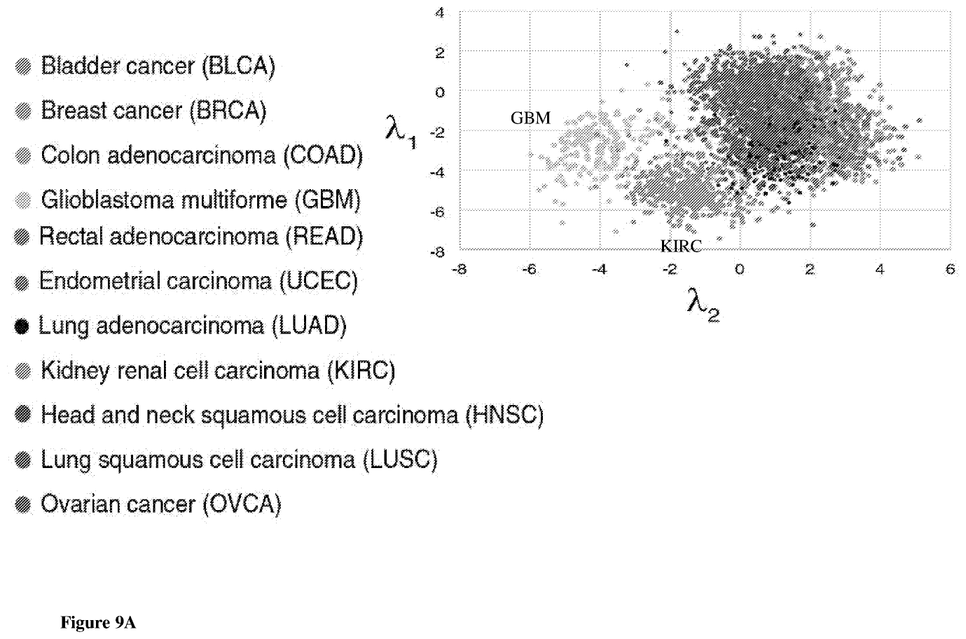

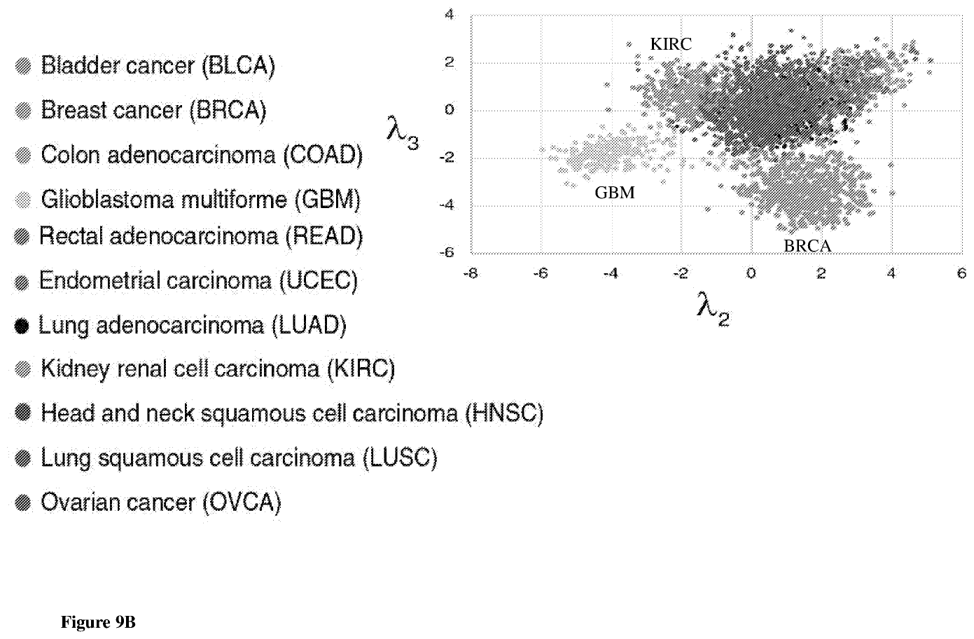

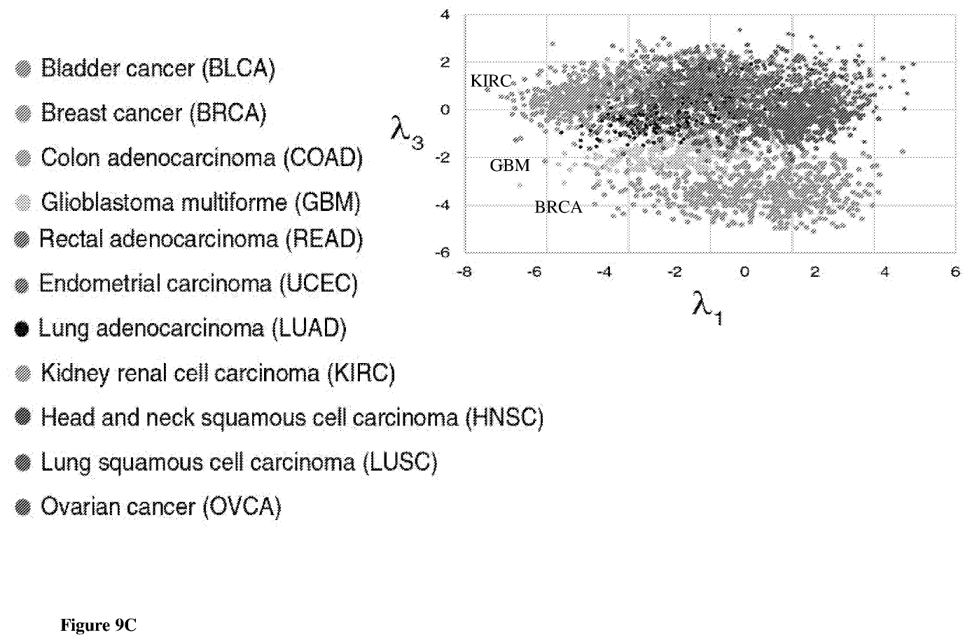

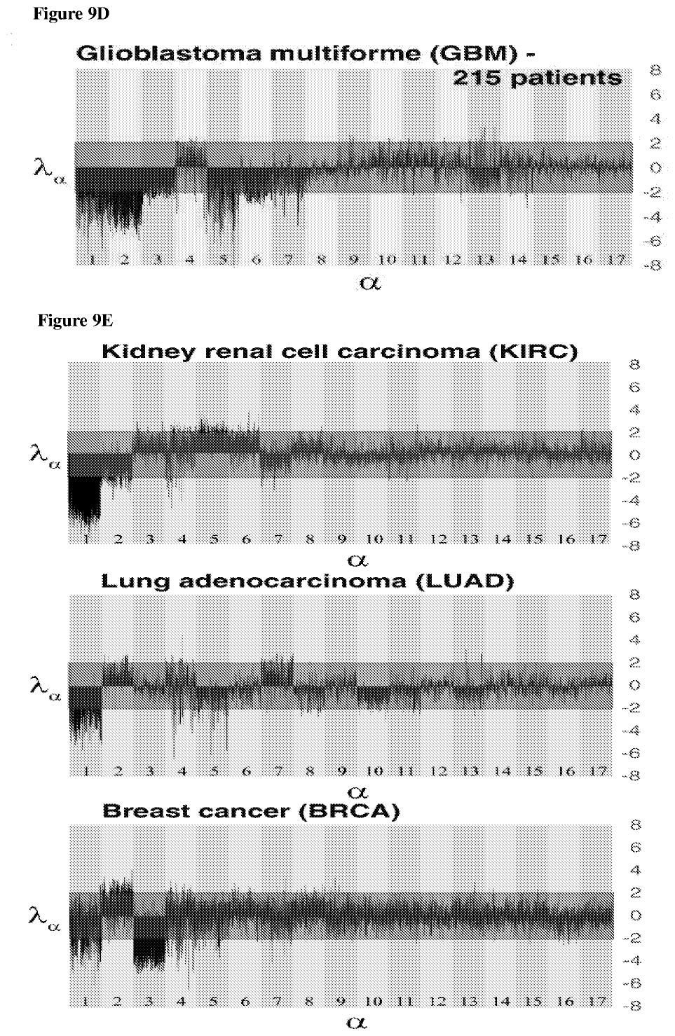

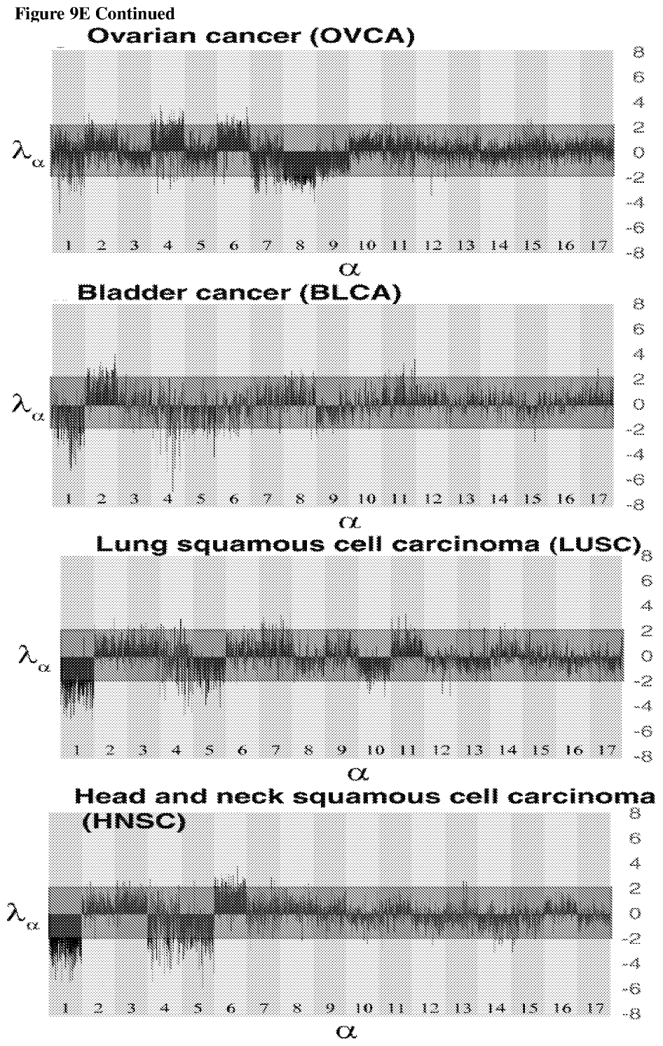

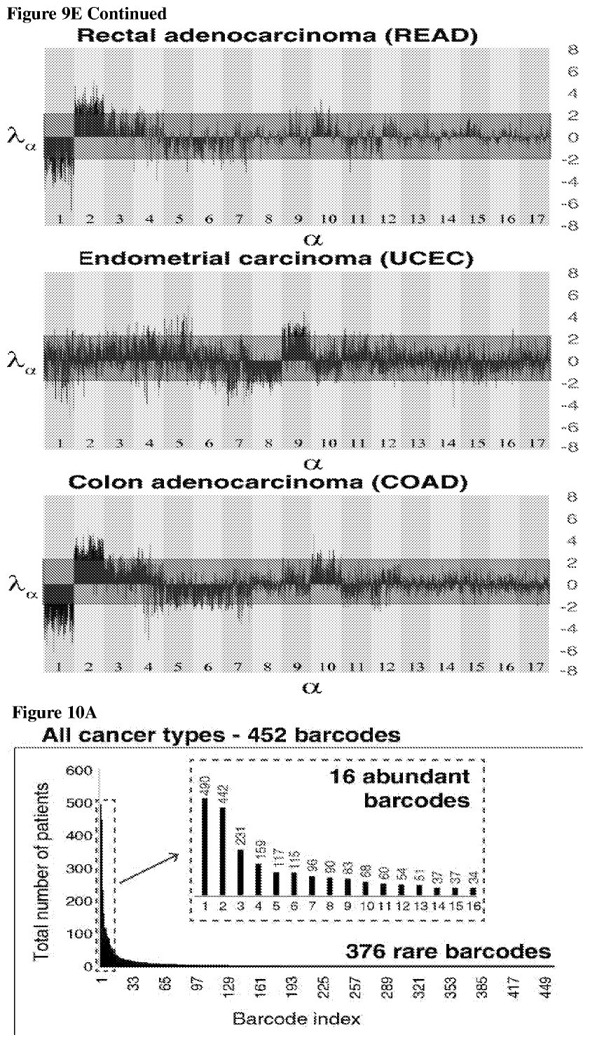

was plotted against the experimental expression level (ln X_{pEGFR}(k)), in 4 different cases: (upper left panel) only the first, most significant unbalanced process was taken into account (n=1); (upper right panel) the 10 most significant unbalanced processes were taken into account (n=10); (lower left panel) the 17 most significant unbalanced processes were taken into account (n=17); (lower right panel) the 21 most significant unbalanced processes were taken into account (n=21). Theoretically, if the experimental values exactly coincide with the theoretical values, all of the points on the graph should fall on the y=x line. Importantly, the processes α=16 and α=17 had significant λ_{α}(k) values (λ_{α}(k)>2 or λ_{α}(k)−2) in some of the patients (see FIG. 9D-E) and thus had to be taken into consideration. The processes α=18, 19 . . . didn't not have significant values in the patients. Moreover, the plot of the data reproduction (n=17) did not change significantly when the processes α=18, 19, 20, 21 . . . were added (see for example n=21). Thus, the plots nicely show how 17 unbalanced processes are enough to reproduce the expression levels of EGFR in all tumors.

17 groups, constructed from the array of 181 proteins and phosphoproteins tested, are enough to describe the biological imbalance that differentiates 3467 tumors (Table 1). Considering the size of the dataset and the variety of tumors it contains, this result is highly interesting. Each tumor is characterized by a specific set of 1-4 unbalanced processes (see below), and therefore 17 unbalanced processes “allow” a very high degree of inter-tumor heterogeneity, because, for example, there are 3213 distinct combinations of 1-4 unbalanced processes that can be chosen out of 17. On the other hand, the fact that each tumor can be portrayed by a small set of unbalanced processes unmasks a surprisingly simple order that underlies the very large complexity of cancer systems.

TABLE 1

| | Proteins and phosphoproteins that participate in the unbalanced process |
|---|---|
| | *The functional connections between the proteins are presented in FIG. 6A. |
| | **The weight of participation (G) is listed in parenthesis for each protein |
| Unbalanced | ***Note that (+/-) signs represent correlation or anti-correlation between |
| process (α) | proteins in the process, and not direction of change in expression level |
| 1 | ACC1 (0.128), CAVEOLIN1 (-0.209), COLLAGENVI (-0.141), CYCLINB1 |
| | (0.221), ECADHERIN (0.103), EGFRPY1068 (-0.285), EGFRPY1173 (-0.138), |
| | ERALPHA (0.166), HER2PY1248 (-0.123), HSP70 (-0.144), |
| | MAPKPT202Y204 (-0.165), S6 (0.139), SRCPY416 (-0.149), PAI1 (-0.112), |
| | FASN (0.205), MYH11 (-0.412), NDRG1PT346 (-0.349), RBM15 (0.136), |
| | TRFC (0.116) |
| 2 | AKTPS473 (-0.231), AKTPT308 (-0.205), CLAUDIN7 (0.280), |
| | ECADHERIN (0.232), EGFRPY1068 (-0.132), GATA3 (0.157), |
| | GSK3ALPHABETAPS21S9 (-0.127), INPP4B (0.213), VEGFR2 (0.116), |
| | EGFR (-0.103), FASN (0.131), GAPDH (-0.191), GSK3PS9 (-0.155), |
| | MYH11 (0.346), NDRG1PT346 (-0.137), RAB25 (0.154), RICTOR (0.142), |
| | VHL (0.317), ACETYLATUBULIN(LYS40) (-0.156) |
| 3 | AR (-0.229), BCL2 (-0.109), BETACATENIN (0.109), |
| | CASPASE7CLEAVEDDI98 (0.156), COLLAGENVI (-0.138), CYCLINB1 |
| | (0.146), CYCLINE1 (0.105), ECADHERIN (0.128), EEF2 (0.150), |
| | ERALPHA (-0.364), FIBRONECTIN (-0.122), GATA3 (-0.329), HSP70 (-0.113), |
| | INPP4B (-0.216), MAPKPT202Y204 (-0.160), FASN (-0.191), |
| | GAPDH (0.327), MYH11 (0.134), NDRG1PT346 (0.111), PEA15PS116 |
| | (0.101), TRANSGLUTAMINASE (0.109), TRFC (0.263) |
| 4 | 53BP1 (0.139), AKTPS473 (0.109), AMPKPT172 (0.110), ATM (0.122), |
| | BETACATENIN (0.288), CASPASE7CLEAVEDD198 (-0.111), CLAUDIN7 |
| | (0.102), ECADHERIN (0.154), EEF2K (0.105), EGFRPY 1068 (0.178), |
| | ERALPHA (0.151), ERK2 (0.115), GAB2 (0.128), |
| | GSK3ALPHABETAPS21S9 (0.149), HER2 (0.149), HSP70 (-0.132), |
| | NFKBP65PS536 (0.159), STAT5ALPHA (0.114), SYK (0.105), TUBERIN |
| | (0.108), PAI1 (-0.185), BRAF (0.130), GAPDH (0.151), GSK3PS9 (0.139), |
| | PDCD4 (0.122), PKCPANBETAIIPS660 (0.135), RBM15 (0.157), VHL |
| | (0.356), ACETYLATUBULIN(LYS40) (0.206) |
| 5 | AKTPS473 (0.103), BCL2 (0.148), BETACATENIN (-0.102), CAVEOLIN1 |
| | (0.100), CLAUDIN7 (-0.125), CYCL1NB1 (-0.184), EGFRPY 1068 (-0.359), |
| | EGFRPY 1173 (-0.196), ERALPHA (0.324), HER2PY1248 (-0.255), IGFBP2 |
| | (-0.179), SRCPY416 (-0.228), PAI1 (-0.146), ASNS (-0.106), EGFR (-0.117), |
| | GAPDH (0.259), MYH11 (0.316), PEA15PS116 (0.105), RICTOR (0.107), |
| | TRFC (-0.105), ETS1 (0.132) |
| 6 | ERALPHA (0.171), ERK2 (-0.110), PKCALPHA (-0.163), |
| | PKCALPHAPS657 (-0.158), S6PS235S236 (0.107), STAT5ALPHA (-0.108), |
| | PAI1 (0.105), GAPDH (0.184), MYH11 (-0.302), NDRG1PT346 (0.426), |
| | PDCD4 (0.124), RICTOR (-0.217), VHL (0.355), |
| | ACETYLATUBULIN(LYS40) (-0.222) |
| 7 | 4EBP1PT37T46 (0.103), AKTPS473 (0.141), AKTPT308 (0.123), AR (-0.113), |
| | CAVEOLIN1 (0.213), EGFRPY1068 (-0.112), EGFRPY1173 (-0.167), |
| | ERALPHA (-0.155), ERK2 (-0.105), GSK3ALPHABETAPS21S9 |
| | (0.144), HSP70 (-0.119), IGFBP2 (-0.145), MAPKPT202Y204 (0.101), |
| | NCADHERIN (-0.129). NFKBP65PS536 (0.122), P38PT180Y182 (0.150), |
| | S6PS235S236 (0.167), S6PS240S244 (0.147), SRCPY527 (0.160), FASN |
| | (0.109), GAPDH (-0.351), GSK3PS9 (0.148), MYH11 (0.181), |
| | NDRG1PT346 (0.106), PDCD4 (0.118), PEA15PS116 (-0.267), ETS1 (-0.120), |
| | ACETYLATUBULIN(LYS40) (-0.184) |
| 8 | ACCPS79 (0.150), ACC1 (0.163), CLAUD1N7 (-0.153), CYCL1NB1 (-0.104), |
| | CYCLINE1 (-0.168), EEF2 (0.143), EGFRPY1068 (0.108), |
| | ERALPHA (-0.215), GAB2 (-0.116), GATA3 (0.213), IGFBP2 (-0.385), |
| | INPP4B (0.176), MAPKPT202Y204 (-0.113), PDK1PS241 (0.109), SYK (-0.141), |
| | VEGFR2 (0.127), FASN (0.303), GAPDH (0.286), MYH11 (-0.165), |
| | PDCD4 (-0.112), RAB25 (0.101), RICTOR (-0.100), |
| | ACETYLATUBULIN(LYS40) (-0.165) |
| 9 | ACCPS79 (0.106), AKTPS473 (0.236), AKTPT308 (0.256), ATM (-0.213), |
| | CMYC (-0.125), CLAUDIN7 (0.226), ECADHERIN (0.181), EEF2 (0.114), |
| | ERALPHA (0.318), ERALPHAPS118 (0.139), FIBRONECTIN (-0.104), |
| | GAB2 (-0.209), GSK3ALPHABETAPS21S9 (0.110), IGFBP2 (0.147), |
| | NFKBP65PS536 (-0.116), PAXILLIN (-0.105), STAT5ALPHA (-0.180), |
| | EIF4G (-0.114), GAPDH (0.112), MYH11 (0.102), RICTOR (-0.119), TRFC |
| | (0.183), VHL (-0.244) |
| 10 | 4EBP1PT37T46 (0.126), AKTPS473 (0.136), BAR (-0.108), BCL2 (-0.105), |
| | CAVEOLINI (-0.172), CLAUDIN7 (0.127), COLLAGENVI (0.110), |
| | ECADHERIN (-0.147), EGFRPY1068 (-0.200), EGFRPY1173 (-0.120), |
| | ERALPHA (-0.168), FIBRONECTIN (0.121), GSK3ALPHABETAPS21S9 |
| | (0.117), HER2PY1248 (-0.108), MAPKPT202Y204 (0.274), P53 (0.101), |
| | P70S6KPT389 (0.130), PAXILLIN (-0.121), RBPS807S811 (0.121), |
| | S6PS235S236 (0.127), SRCPY527 (0.183), BRAF (-0.122), EIF4G (-0.101), |
| | GAPDH (0.147), PDCD4 (0.117), PEA15PS116 (0.381), TIGAR (0.113), |
| | TRANSGLUTAMINASE (-0.147), VHL (0.158), ETS1 (0.129) |
| 11 | ACC1 (0.171), BETACATENIN (-0.102), CAVEOLIN1 (0.148), CLAUDIN7 |
| | (-0.174), CYCLINB1 (0.200), ECADHERIN (-0.305), ERALPHA (0.124), |
| | FIBRONECTIN (0.156), HER3 (-0.107), IGFBP2 (0.322), PAI1 (0.249), |
| | ASNS (0.138), FASN (0.190), GAPDH (0.242), MYH11 (0.203), PDCD4 (-0.161), |
| | PEA15PS 116 (-0.132), RAB25 (-0.108), RICTOR (0.165), TRFC |
| | (0.206), VHL (0.191), ACETYLATUBULIN(LYS40) (0.140) |
| 12 | ACCPS79 (0.105), ACC1 (0.142), ATM (0.170), CAVEOLIN1 (-0.223), |
| | CLAUDIN7 (0.140), EEF2 (-0.152), FIBRONECTIN (-0.123), HER2 (-0.169), |
| | HER2PY1248 (-0.114), IGFBP2 (-0.216), NFKBP65PS536 (-0.108), |
| | P53 (0.106), PAXILLIN (-0.153), PKCALPHAPS657 (0.133), S6 (0.102), |
| | SHCPY317 (0.103), SRCPY527 (0.109), SYK (0.148), YB1 (-0.169), ASNS |
| | (0.144), FASN (0.123), MYH11 (0.103), NDRG1PT346 (0.108), |
| | PEA15PS116 (-0.256), PRDX1 (0.109), RAB25 (0.178), TRFC (-0.162), |
| | TSC1 (0.109), ETS1 (-0.167) |
| 13 | AKTPS473 (0.106), AMPKPT172 (-0.103), BAX (-0.112), BETACATENIN |
| | (-0.156), CMYC (0.122), CASPASE7CLEAVEDD198 (-0.119), |
| | CAVEOLIN1 (-0.111), CYCLINB1 (0.104), EGFRPY1068 (0.412), |
| | EGFRPY1173 (0.141), ERK2 (-0.102), HER2PY1248 (0.125), IGFBP2 (-0.108), |
| | INPP4B (-0.165), MAPKPT202Y204 (-0.140), P38PT180Y182 (-0.109), |
| | P53 (0.113), PEA15 (-0.131), PTEN (-0.127), SHCPY317 (0.118), |
| | SYK (-0.199), PAI1 (-0.173), FASN (0.129), FOXM1 (0.117), MYH11 |
| | (0.256), NDRG1PT346 (-0.129), PKCPANBETAIIPS660 (-0.127), RICTOR |
| | (0.173), VHL (0.166), ACETYLATUBULIN(LYS40) (-0.117) |
| 14 | AKTPS473 (0.120), AKTPT308 (0.142), ATM (-0.141), |
| | CASPASE7CLEAVEDD198 (-0.233), CAVEOLIN1 (-0.262), CLAUDIN7 |
| | (0.129), ECADHERIN (0.140), EEF2 (-0.111), EGFRPY1068 (-0.287), |
| | EGFRPYI173 (-0.134), ERALPHA (-0.285), HER2PYI248 (-0.148), |
| | IGFBP2 (0.127), MAPKPT202Y204 (-0.124), P38PT180Y182 (-0.125), |
| | RAD50 (-0.113), S6PS235S236 (-0.160), S6PS240S244 (-0.125), SRCPY416 |
| | (-0.103), SYK (-0.121), BRAF (0.124), MYH11 (0.121), NDRG1PT346 |
| | (0.258), PDCD4 (-0.149), PEA15PSI16 (-0.117), |
| | ACETYLATUBULIN(LYS40) (0.231) |
| 15 | 4EBP1 (0.103), 4EBP1PT37T46 (0.133), AKTPS473 (-0.199), AKTPT308 (-0.263), |
| | CAVEOLIN1 (-0.115), CYCLINB1 (0.142), GAB2 (0.113), GATA3 |
| | (0.124), HER2 (0.111), IGFBP2 (0.111), NFKBP65PS536 (0.109), |
| | PKCALPHA (-0.119), PTEN (0.106), RBPS807S811 (0.120), FOXM1 |
| | (0.100), GAPDH (0.241), MYH11 (0.273), NDRG1PT346 (0.120), |
| | PEA15PS116 (0.109), RAB25 (-0.131), TRANSGLUTAMINASE (-0.141), |
| | VHL (-0.491), ACETYLATUBULIN(LYS40) (-0.182), P62LCKLIGAND |
| | (0.132) |
| 16 | AKTPS473 (-0.213), AKTPT308 (-0.159), CASPASE7CLEAVEDD198 (-0.358), |
| | CAVEOLIN1 (0.340), HER2 (-0.134), HSP70 (-0.206), LCK (-0.159), |
| | PKCALPHA (0.117), S6PS240S244 (-0.131), SRCPY527 (0.163), |
| | STAT5ALPHA (-0.172), SYK (-0.210), VEGFR2 (0.117), YAPPS127 |
| | (0.105), GAPDH (0.267), MYH11 (-0.155), NDRG1PT346 (-0.120), |
| | PEA15PS116 (-0.105), RAB25 (0.109), RICTOR (0.163), |
| | TRANSGLUTAMINASE (-0.125), P62LCKLIGAND (-0.142) |
| 17 | AMPKPT172 (-0.202), BCL2 (-0.170), BIM (-0.102), |
| | CASPASE7CLEAVEDD198 (-0.143), ECADHERIN (0.119), |
| | FIBRONECTIN (0.129), HER2 (0.139), IGFBP2 (-0.118), INPP4B (0.213), |
| | NFKBP65PS536 (-0.112), S6 (0.137), S6PS235S236 (0.317), S6PS240S244 |
| | (0.294), SRCPY416 (-0.101), SRCPY527 (-0.197), YAPPS127 (0.160), PAI1 |
| | (0.207), G6PD (0.200), NDRG1PT346 (-0.164), PDCD4 (0.112), |
| | TRANSGLUTAMINASE (0.218), VHL (-0.157), P62LCKLIGAND (0.106) |

Our approach is based on analyses of multiple-patient data. Therefore, a relevant question that may arise is how large the dataset should be in order to obtain accurate results. To address this question, we randomly picked 100 patients from each type of cancer (1100 patient total, representing about a third of the complete dataset), and the same analysis was performed on this smaller matrix (FIG. 7A-B). λ_{α} values for 3 random patients are shown in FIG. 7A respectively, demonstrating that their diagnoses remain essentially the same when only 1100 tumors were used for the analysis instead of 3467. Gᵢ₁ values obtained from the analysis of the small matrix were plotted against Gᵢ₁ values obtained from the analysis of the original matrix, and the results were found to be very similar (FIG. 7B). Thus, with either matrix size the patient diagnoses remained essentially the same.

### Example 9: Each Tumor in the Dataset Harbors a Set of 1-4 Unbalanced Molecular Processes (Proteomics Dataset)

Our analysis revealed that the tumors in the dataset are each characterized by a combination of 1-4 unbalanced processes out of 17. Two examples for each cancer type are provided in Table 2. The variety of combinations of unbalanced processes that appear in the different tumors is what underlies the disparities in protein expression levels between different patients. Note that the different unbalanced processes may each represent a number of signaling pathways, some of them rewired, that have deviated from the balanced state in a coordinated manner, e.g. one pathway can be upregulated and the other downregulated, both can be upregulated together, etc. This is an important attribute of surprisal analysis, because it simplifies the design of therapy for every tumor: While a specific tumor may demonstrate aberrations in multiple signaling pathways, these pathways may change in a coordinate manner and thus be represented by a smaller number of unbalanced processes. We hypothesize that targeting one central hub protein from each unbalanced process will be enough to reduce the patient-specific signaling imbalance.

TABLE 2

| | | Combination | |
|---|---|---|---|
| | | of unbalanced | |
| Patient | Tumor | processes | Patient-specific barcode |
| index | type | in the tumor | (λ*₁, λ*₂, λ*₃, . . . , λ*₁₇) |
| 2 | BLCA | 1 (negative); | [ −1. 0. 0. 0. 0. −1. 0. 0. −1. 0. 1. |
| | | 6 (negative); | 0. 0. 0. 0. 0. 0.] |
| | | 9 (negative); | |
| | | 11 (positive); | |
| 3 | BLCA | 2 (positive); | [ 0. 1. 0. 0. 0. 0. 0. 0. 0. 0. 0. 0. |
| | | | 0. 0. 0. 0. 0.] |
| 1001 | COAD | 2 (positive); | [ 0. 1. 0. 0. −1. 0. 0. 0. 0. 0. 0. 0. |
| | | 5 (negative); | 0. 0. 0. 0. 0.] |
| 1100 | COAD | 1 (negative); | [ −1. 1. 0. 0. 0. 0. 0. 0. 0. 0. 0. 0. |
| | | 2 (positive); | 0. 0. 0. 0. 0.] |
| 1222 | GBM | 2 (negative); | [ 0. −1. 0. 0. 0. 0. 0. 0. 0. 0. 0. 0. |
| | | | 0. 0. 0. 0. 0.] |
| 1380 | GBM | 1 (negative): | [ −1. −1. −1. 0. −1. 0. 0. 0. 0. 0. |
| | | 2 (negative); | 0. 0. 0. 0. 0. 0. 0.] |
| | | 3 (negative); | |
| | | 5 (negative); | |
| 1434 | HNSC | 5 (negative); | [ 0. 0. 0. 0. −1. −1. 0. 0. 0. 0. 0. |
| | | 6 (positive); | 0. 0. 0. 0. 0. 0.] |
| 1602 | HNSC | 6 (positive); | [ 0. 0. 0. 0. 0. 1. 0. 0. 0. 0. 0. 0. |
| | | | 0. 0. 0. 0. 0.] |
| 1768 | KIRC | 1 (negative); | [ −1. −1. 0. 0. 1. 0. 0. 0. 0. 0. 0. |
| | | 2 (negative); | 0. 0. 0. 0. 0. 0.] |
| | | 5 (positive); | |
| 1852 | KIRC | 1 (negative); | [ −1. −1. 0. 0. 0. 0. 0. 0. 0. 0. 0. |
| | | 2 (negative); | 0. 0. 0. 0. 0. 0.] |
| 2208 | LUAD | 1 (negative); | [ −1. 0. 0. 4. 0. 0. 0. 0. 0. 0. 0. 0. |
| | | 4 (positive); | 0. 0. 0. 0. 0.] |
| 2210 | LUAD | 1 (negative); | [ −1. 0. 0. 0. 0. 0. 1. 0. 0. 0. 0. 0. |
| | | 7 (positive); | 0. 0. 0. 0. 0.] |
| 2331 | LUSC | 1 (negative); | [ −1. 0. 0. −1. 0. 0. 0. 0. 0. 0. 0. |
| | | 4 (negative); | 0. 0. 0. 0. 0. 0.] |
| 2335 | LUSC | 1 (negative); | [ −1. 0. 0. 1. 0. 0. 0. 0. 0. 0. 0. 0. |
| | | 4 (positive); | 0. 0. 0. 0. 0.] |
| 2523 | OVCA | 8 (negative); | [ 0. 0. 0. 0. 0. 0. 0. −1. 0. 0. 0. 0. |
| | | | 0. 0. 0. 0. 0.] |
| 2524 | OVCA | 1 (negative); | [ −1. 1. 0. 0. 0. 0. 0. −1. 0. 0. 1. |
| | | 2 (positive); | 0. 0. 0. 0. 0. 0.] |
| | | 8 (negative); | |
| | | 11 (positive); | |
| 2943 | READ | 1 (negative); | [ −1. 1. 0. 0. 0. 0. 0. 0. 0. 1. 0. 0. |
| | | 2 (positive); | 0. 0. 0. 0. 0.] |
| | | 10 (positive); | |
| 2944 | READ | 1 (negative); | [ −1. 0. 0. 0. 0. 0. 0. 0. 0. 0. 0. 0. |
| | | | 0. 0. 0. 0. 0.] |
| 3096 | UCEC | 4 (positive); | [ 0. 0. 0. 1. 0. 0. 0. 0. 1. 0. 0. 0. |
| | | 9 (positive); | 0. 0. 1. 0. 0.] |
| | | 15 (negative); | |
| 3097 | UCEC | 4 (positive); | [ 0. 0. 0. 1. 0. 0. 0. 0. 1. 0. 0. 0. |
| | | 9 (positive); | 0. 0. 0. 0. 0.] |

### Example 10: Surprisal Analysis Provides a Patient-Explicit, Often Rewired, Network Structure (Proteomics Dataset)

Tumors frequently harbor protein networks that have undergone significant rewiring. The process of protein network rewiring is dependent on the molecular and environmental context and is therefore tumor-specific. The ability to decipher patient-specific protein network structures in an accurate manner is crucial to the design of patient-tailored medicine.

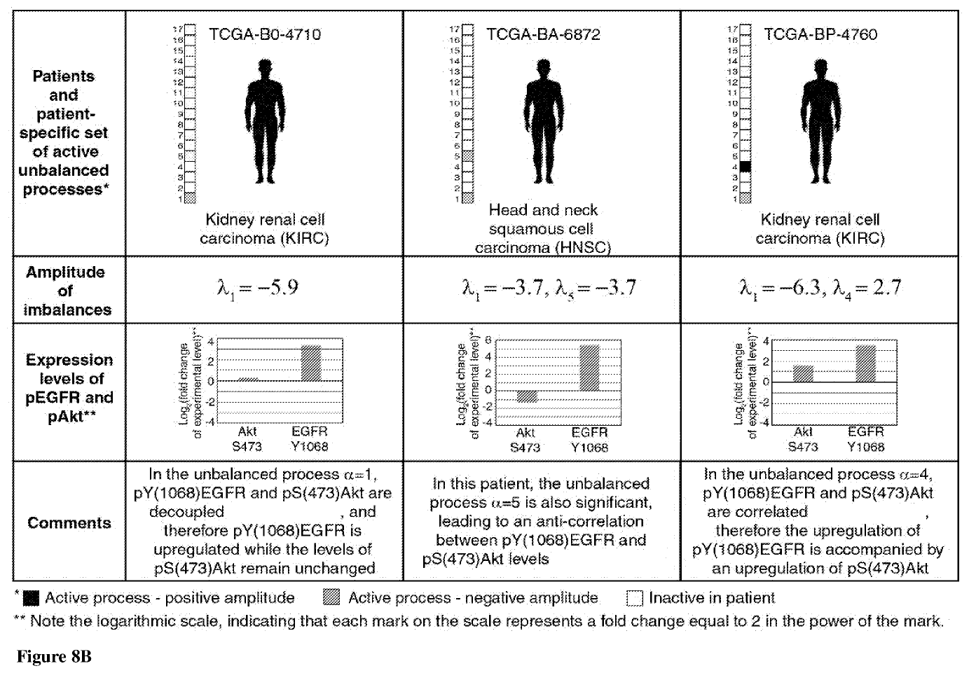

We examined EGFR signaling in the 17 unbalanced processes. The activated form of EGFR, phosphorylated on Y1068 and/or Y1173, was significantly influenced by 7 of the 17 unbalanced processes that we identified in the 3467 tumors: 1, 4, 5, 7, 10, 13, 14 (FIG. 8A). Noticeably, the 7 different unbalanced networks in which EGFR participates are very different from each other (FIG. 6A). In all of these networks EGFR is a central hub, however, significant rewiring of the signaling pathway occurred. For example, unbalanced process 1 does not influence the levels of pS(473)Akt, one of the major downstream effectors of EGFR (FIG. 6A). This is exceptionally interesting, considering that unbalanced process 1 is the most significant process, appearing as significant in 48.3% of the whole population of 3467 tumors. Furthermore, in unbalanced process 5 ppEGFR (pY(1068)EGFR and pY(1173)EGFR) and pS(473)Akt are anti-correlated (FIG. 6A). This means that in tumors for which A, is significant and positive, this process leads to upregulation of pS(473)Akt and downregulation of ppEGFR (see Methods). In tumors where such an anti-correlation exists, inhibition of EGFR in order to inhibit signaling to Akt would be useless. EGFR and Akt are anti-correlated in the unbalanced processes 7, 10, and 14 (FIG. 6A) as well, appearing in 24.6% of the 3467 tumors. In contrast, EGFR and Akt are correlated in the unbalanced processes 4 and 13 (FIG. 6A; appearing in 16.6% of the tumors) and non-correlated (pS(473)Akt is absent) in unbalanced process 1 (appearing in 48.3% of the tumors). FIG. 8B shows 3 representative patients for which the EGFR signaling pathway has undergone such significant rewiring.

Another major downstream effector of ppEGFR is pT(202)Y(204)MAPK. Indeed, our analysis revealed that ppEGFR and pT(202)Y(204)MAPK are correlated in the unbalanced processes 1 and 14 (FIG. 6A; appearing in 49.3% of the tumors). However, they are anti-correlated in the unbalanced processes 7, 10 and 13 (appearing in 12.2% of the tumors), and non-correlated (pT(202)Y(204)MAPK is absent) in the unbalanced processes 4 and 5 (appearing in 15.5% of the tumors).

This ability to accurately analyze the reorganization of protein network structures in individual tumors is one of the most powerful attributes of our analysis, which differs it from other computational techniques, and forms the basis for the efficient design of patient-explicit combination therapies.

### Example 11: Utilizing the Complete Sets of Patient-Specific Unbalanced Processes, as Identified by Surprisal Analysis, is Essential for the Efficient Mapping of 3467 Patients (Transcriptomics Dataset)

We wished to utilize the comprehensive data obtained by surprisal analysis for the development of a simple method to design patient-explicit combination therapies. To this end, we sought to achieve efficient mapping of the 3467 patients.

We examined the weights of the unbalanced processes, λ₁(k), λ₂(k), and λ₃(k) (FIG. 9A-N). These are the three most significant unbalanced processes, each appearing in over 25% of the tumors, and including a number of notorious cancer-associated proteins such as p-EGFR, p-Src, p-Akt, estrogen receptor α (ERα), androgen receptor (AR) and more (FIG. 6A). Some cancer types are nicely separated, e.g. GBM, KIRC and BRCA by λ₂ (FIG. 9A-B) and λ₃ (FIG. 9B-C). However, though some cancer types can be separated to some extent according to these 3 processes, representation of tumors using these 2D graphs does not enable the accurate mapping of individual tumors. Many tumors with distinct protein network structures appear in the same place in these graphs, e.g. most UCEC and HNSC tumors, which we found to be highly heterogeneous (see below) (FIG. 9A-C). These results demonstrate that a partial identification of unbalanced molecular processes does not suffice to accurately map the cancer patients.

We inspected each type of tumor individually, aiming to study whether there are specific sets of unbalanced processes that are characteristic of specific tumor types. For example, in FIG. 9D we present the amplitudes, λ_{α}(k), of all 17 unbalanced processes in the entire population of GBM tumors. The majority of GBM tumors display a signature comprising a subset of the unbalanced processes 1, 2, 3, 5, 6 (FIG. 9D), demonstrating some degree of cancer type-specific commonality. Note that for every process, some tumors demonstrate λ_{α}(k)≈0, and are not seen on the graph; e.g. λ₁(k) is not necessarily negative in all GBM tumors tested. Additionally, for most unbalanced processes, there is a considerable number of tumors for which the specific process is insignificant (the gray box marks the threshold limits). Importantly, some unbalanced processes were rarely or differentially expressed. For example, λ₁(k) appeared significant only in a minority of GBM tumors, while λ₁(k) was heterogeneously expressed. These results demonstrate that while there are unbalanced processes common to many GBM tumors, the majority of GBM tumors are not alike, when looking at the complete set of tumor-specific unbalanced processes. This notion is especially important when devising a treatment for GBM: The entire tumor-specific unbalanced network needs to be targeted in order to effectively treat the tumor. The same analysis was also performed for the other tumor types (FIG. 9E).

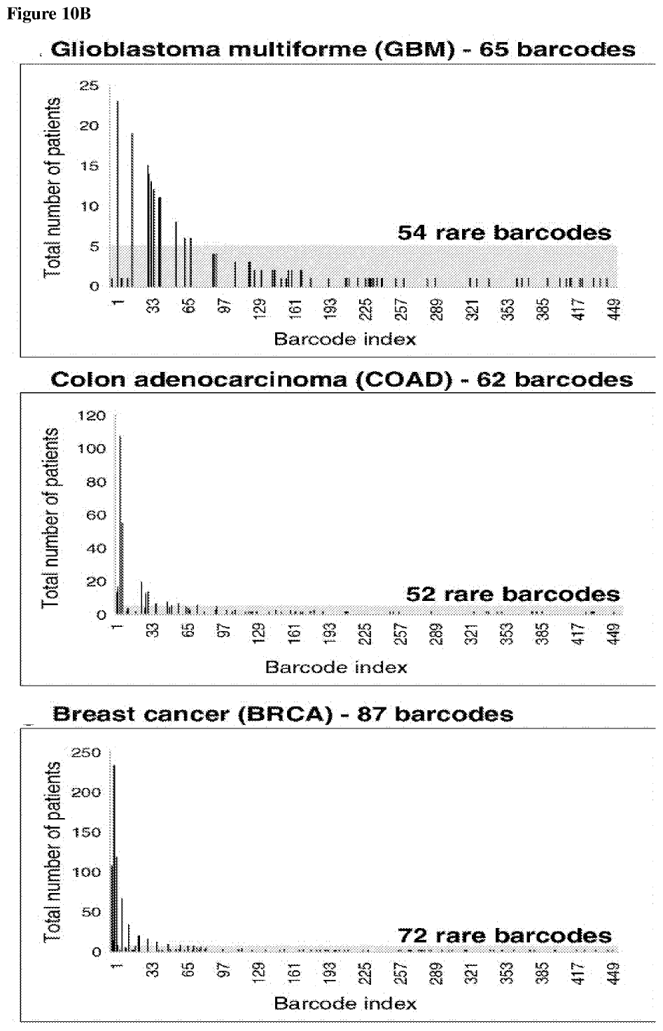

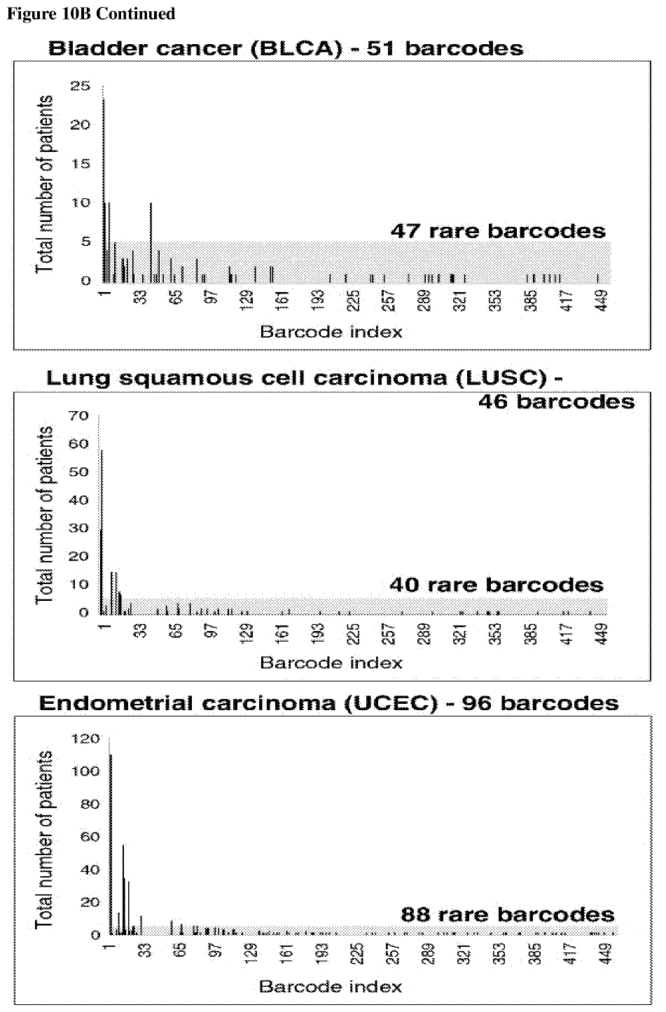

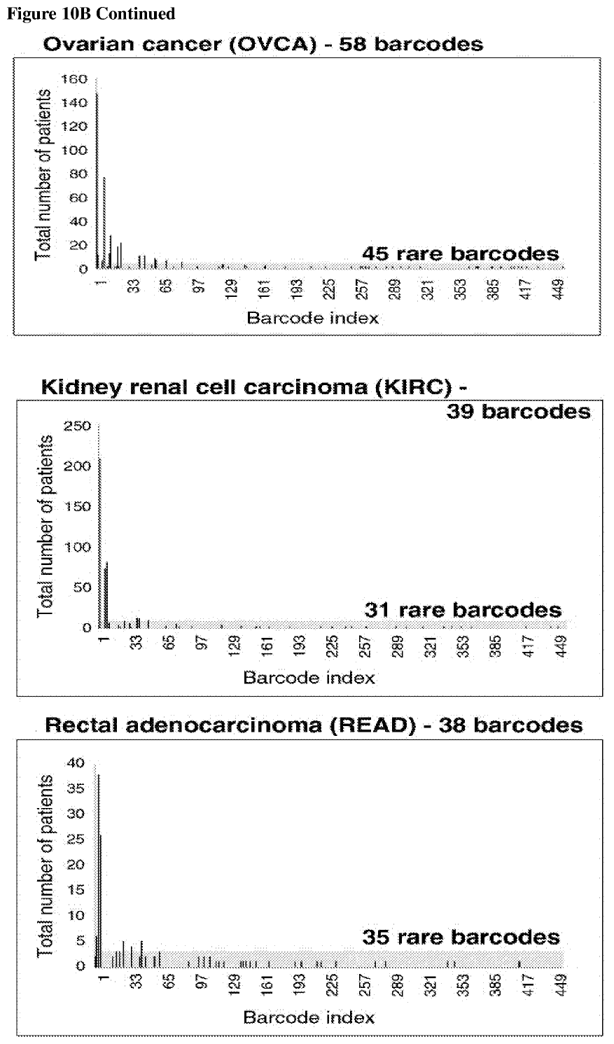

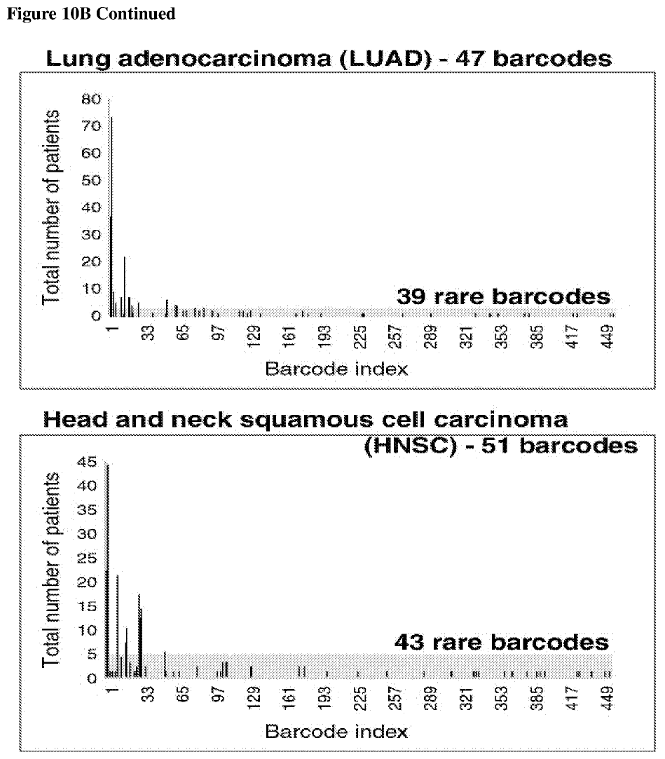

Next, we looked into patient-explicit signatures of processes. To study the recurrence of the different unbalanced processes in the different tumors, we defined λ*_{α}(k), which can hold one of three values: −1, 0, or 1. λ*_{α}(k)=±1 represents significant amplitudes (i.e. λ_{α}(k) exceed threshold limits, see Methods), whereas λ*_{α}(k)=0 represents insignificant amplitudes. Therefore, the λ*_{α}(k) values define a specific barcode for each individual tumor, which indicates the unbalanced processes that influence it and their signs, disregarding their precise amplitudes. This way the entire collection of tumor-specific sets of unbalanced processes can be compared to one another. Examples of barcodes for 2 patients from each cancer type can be found in Table 2. We found that 452 distinct barcodes repeat themselves in the 3467 tumors. Interestingly, while 16 barcodes were relatively abundant (i.e. each represent 1% or more of the population of tumors), most barcodes were extremely rare: 376 barcodes each represent only 5 tumors or less. 273 of these barcodes represent only a single patient each (FIG. 10A). These rare barcodes were distributed across all 11 cancer types (FIG. 10B). In order to assign the correct therapy to all of these patients, it is vital to inspect each of the tumors individually and accurately.

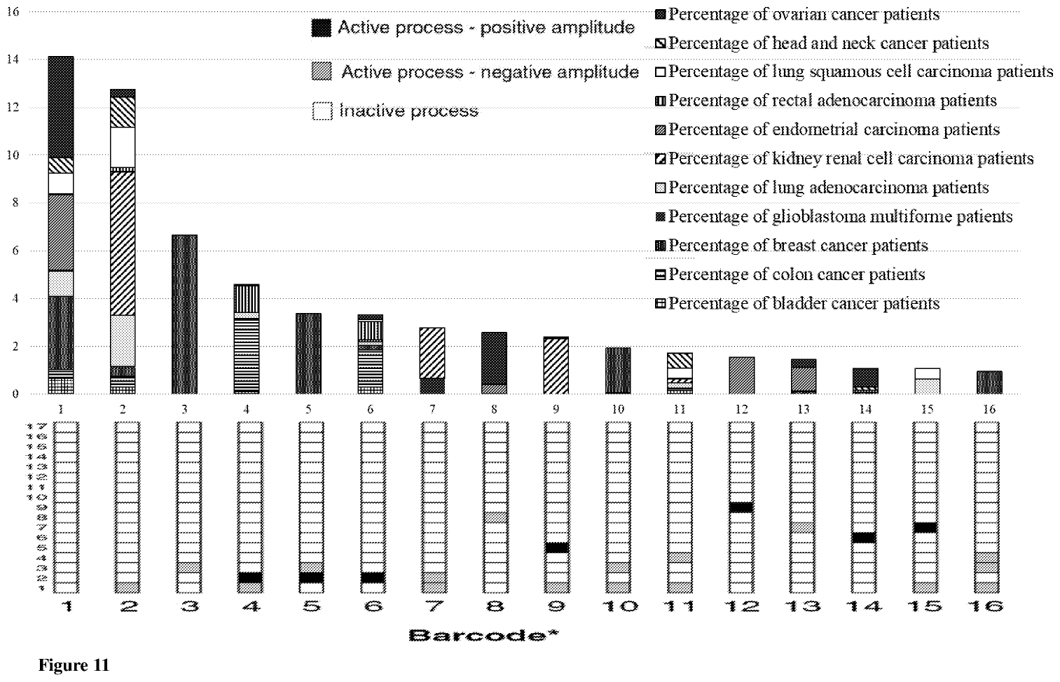

Contrary to the existing methods to classify cancer patients, the representation of tumors according to the barcode of unbalanced processes that they possess enables precisely mapping each and every patient (FIG. 11). Transformation of the proteomic data obtained from tumors into a 17-dimensional space, represented by 17 distinct unbalanced processes, allows to unambiguously map every single patient into this space, including patients that harbor one-of-a-kind tumors. We demonstrate this in FIG. 11 for the 16 most abundant barcodes (each representing at least 35 patients). Each column in the graph represents a specific barcode of unbalanced processes, color-coded according to the different cancer types that possess this barcode. Once the data is represented this way, a wealth of information can be extracted. For example, it is evident that most of the barcodes represent multiple cancer types, i.e. most bars contain multiple colors. Additionally, each cancer type is represented by multiple barcodes, i.e. each color appears in multiple bars in the graph.

Notably, the most abundant barcode (indexed 1 in FIG. 11 and appearing in 14.1% of tumors) is the null barcode, containing no unbalanced processes (FIG. 11). This means that tumors harboring this barcode are non-heterogeneous in the pathways captured by the current array of proteins. The representation of the data as shown in FIG. 11 allows to gain important insights into this finding. For example, none of the GBM tumors in the dataset were assigned the null barcode, suggesting that the protein array tested provides sufficient coverage for the molecular processes that are heterogeneously altered in GBM malignancies. The unbalanced processes in the KIRC tumors in the dataset were also fully covered by the array. On the contrary, 35.7% of OVCA patients and 27% of ECUC patients were assigned the null barcode, suggesting that these tumors do not differentially express the proteins tested (FIG. 11).

Another interesting observation is that, for example, most GBM patients are not represented in the graph in FIG. 4. While ˜10% of GBM patients are represented by barcode 7 (FIG. 11), most GBM patients are each represented by a rare barcode. This finding is true for all tumor types—out of the collection of barcodes that represent the specific population of tumors, the vast majority of barcodes are rare, i.e. appear only in 5 tumors or less. 47 of the 51 barcodes in BLCA tumors are rare; 54 of the 65 barcodes that represent GBM tumors are rare; and 40 of the 46 barcodes in LUSC are rare. In general, at least 78% of the barcodes were rare in each of the tumor type tested (FIG. 10B).

Another interesting finding is related to BRCA tumors. Barcodes 3, 5, and 10 represent almost invariably BRCA patients (FIG. 11). These barcodes contain the unbalanced processes 1, 2 and 3, that include oncogenes such as estrogen receptor α (ERα), androgen receptor (AR), EGFR, VEGFR2, and more (FIG. 6A). However, not all BRCA patients are represented by these three barcodes. In order to precisely map all BRCA patients, the complete set of unbalanced process should be examined. For example, there is a single BRCA patient that harbors a barcode, containing the unbalanced processes 1, 4, and 7. Another one-of-a-kind BRCA patient harbors barcode 73, containing the unbalanced processes 2 and 7. Many more BRCA patients may be overlooked unless their tumors are analyzed in terms of the complete array of 17 unbalanced processes.

Genomic analysis is routinely used in clinics, in order to determine the pathological state of tumors and to assign therapy to the patient. Accumulating evidence from laboratories around the world show that a multi-omics approach, rather than a genomic one alone, is needed in order to correctly resolve oncogenic alterations. We randomly checked pairs of patients with the same proteomic phenotype, i.e. the same barcode in this dataset, and found that their genomic profiles differ significantly (according to the TCGA database). This further underscores the need for a multi-omics approach to cancer therapy.

### Example 12: Suggesting Patient-Explicit Combination Therapies

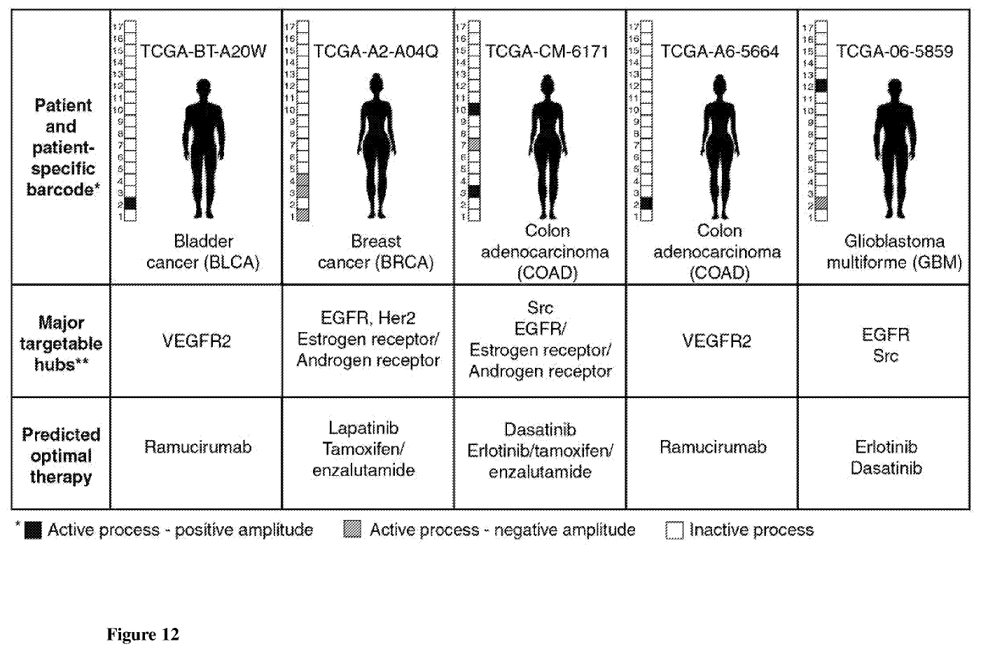

Next, we used our results to suggest the optimal combination of drugs for each patient, which is predicted to reduce the inter-tumor heterogeneity. Each unbalanced process was examined individually, the major targetable hubs were chosen, and FDA-approved drugs were assigned accordingly to each process (Table 3). Then, each patient was assigned a combination of drugs according to the specific signature of unbalanced process that his tumor harbors. FIG. 12 exemplifies this process for 5 patients. Importantly, various drug combinations were suggested for different tumors of the same type. For example, until today only one targeted therapy was approved for the treatment of BLCA (Atezolizumab, an antibody against programmed cell death-ligand 1 (PD-L1)). Our analysis suggests that BLCA patients can be treated with combinations that in many cases include erlotinib (an EGFR inhibitor, approved for the treatment of lung and pancreatic cancer), ramucirumab (an antibody against VEGFR2, approved for the treatment of colon cancer, adenocarcinoma of the stomach and lung cancer) and tamoxifen (an estrogen receptor inhibitor, approved for the treatment of breast cancer). Another example is HNSC: Targeted therapies for the treatment of HNSC include anti-PD-1 and anti-EGFR antibodies. Our analysis indeed suggested combination therapies encompassing erlotinib for the majority of HNSC patients. However, the suggested combinations for the HNSC patients in the dataset also frequently include FDA-approved drugs such as tamoxifen and dasatinib (a Src/Abl dual inhibitor, approved for the treatment of chronic myelogeneous leukemia and acute lymphoblastic leukemia).

TABLE 3

| Unbalanced | | | |
|---|---|---|---|
| process | λ_{α}(k) | Major targetable hubs | Suggested drugs |
| 1 | positive | Estrogen receptor | tamoxifen |
| | negative | EGFR | erlotinib |
| 2 | positive | VEGFR2 | ramucirumab |
| | negative | EGFR | erlotinib |
| 3 | positive | * | * |
| | negative | Estrogen receptor, | tamoxifen/enzalutamide |
| | | androgen receptor | |
| 4 | positive | EGFR, Her2 | lapatinib |
| | negative | * | * |
| 5 | positive | Estrogen receptor | tamoxifen |
| | negative | EGFR, Src | erlotinib/dasatinib |
| 6 | positive | Estrogen receptor | tamoxifen |
| | negative | * | * |
| 7 | positive | Src | dasatinib |
| | negative | EGFR, Estrogen receptor, | erlotinib/tamoxifen/ |
| | | Androgen receptor | enzalutamide |
| 8 | positive | EGFR, VEGFR2 | erlotinib/ramucirumab |
| | negative | Estrogen receptor | tamoxifen |
| 9 | positive | Estrogen receptor | tamoxifen |
| | negative | * | * |
| 10 | positive | Src | dasatinib |
| | negative | EGFR, estrogen receptor, | erlotinib/tamoxifen/ |
| | | Braf | vemurafenib |
| 11 | positive | Estrogen receptor | tamoxifen |
| | negative | * | * |
| 12 | positive | Src | dasatinib |
| | negative | Her2 | trastuzumab |
| 13 | positive | EGFR | erlotinib |
| | negative | * | * |
| 14 | positive | Braf | vemurafenib |
| | negative | EGFR, estrogen receptor, | erlotinib/tamoxifen/ |
| | | Src | dasatinib |
| 15 | positive | Her2 | trastuzumab |
| | negative | * | * |
| 16 | positive | VEGFR2, Src | Ramucirumab/dasatinib |
| | negative | Her2 | trastuzumab |
| 17 | positive | Her2 | trastuzumab |
| | negative | Src | dasatinib |

### Example 13: A Proposed Approach for Personalized Cancer Therapy (Proteomics Dataset)

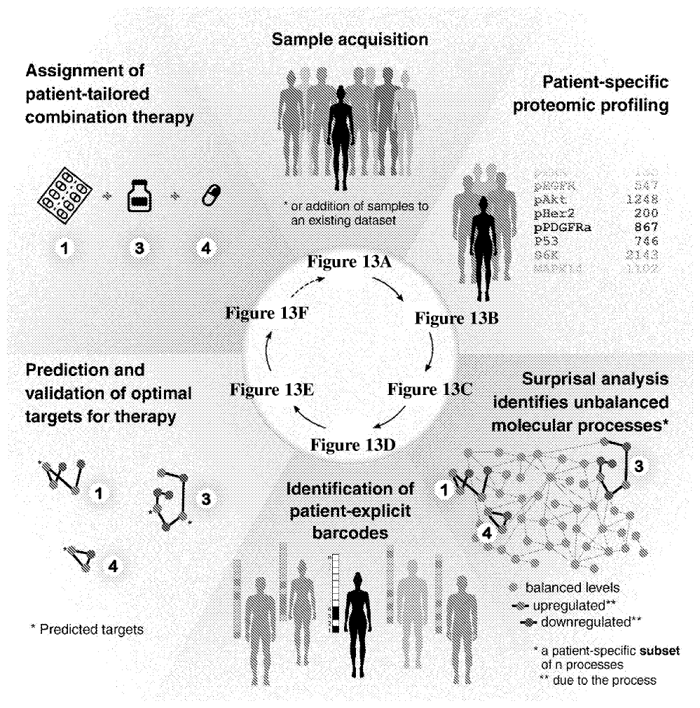

Based on our results, we propose the following approach for the development of a patient-explicit cancer therapy regimen: Following acquisition of tumor samples (FIG. 13A), proteomic profiling will be performed for all samples (FIG. 13B). The data will be analyzed computationally utilizing surprisal analysis and compared to matching non-cancer control samples, in order to identify the unbalanced molecular processes that have emerged in each and every tumor (FIG. 13C). Once the patient-explicit barcodes have been elucidated (FIG. 13D), the optimal drug targets will be predicted and tested experimentally (FIG. 13E), and then a patient-tailored combination therapy will be assigned to the patients (FIG. 13F). We hypothesize that targeting the patient-explicit heterogeneous signaling imbalances, in conjunction with a chemo- or immunotherapy will be the most effective treatment. Once we obtain a large enough database, additional patients/samples can be added to the analysis individually, without the need to collect multiple samples at once.

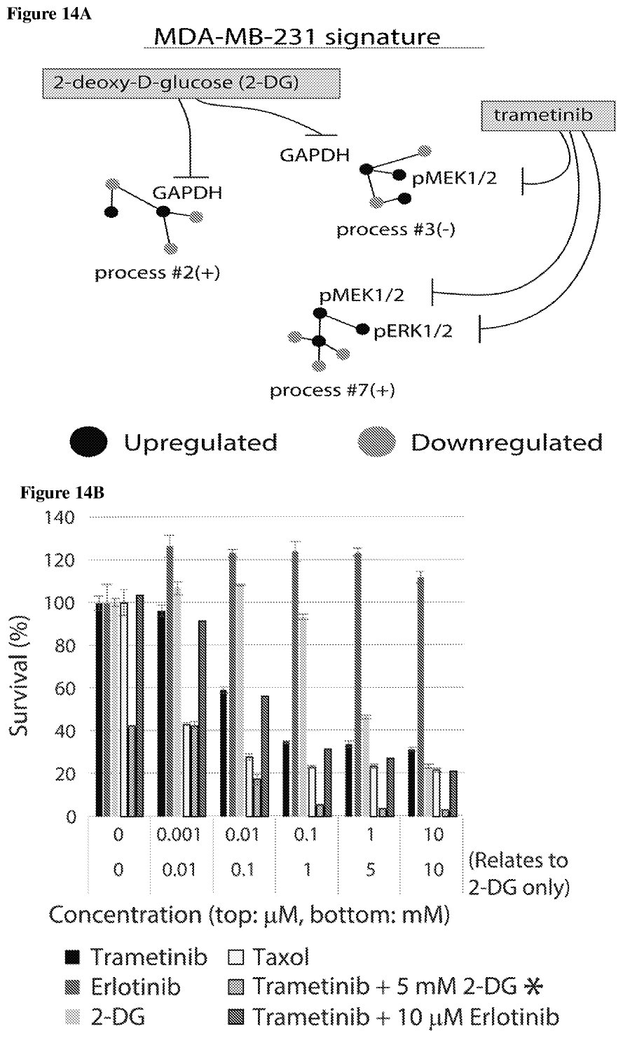

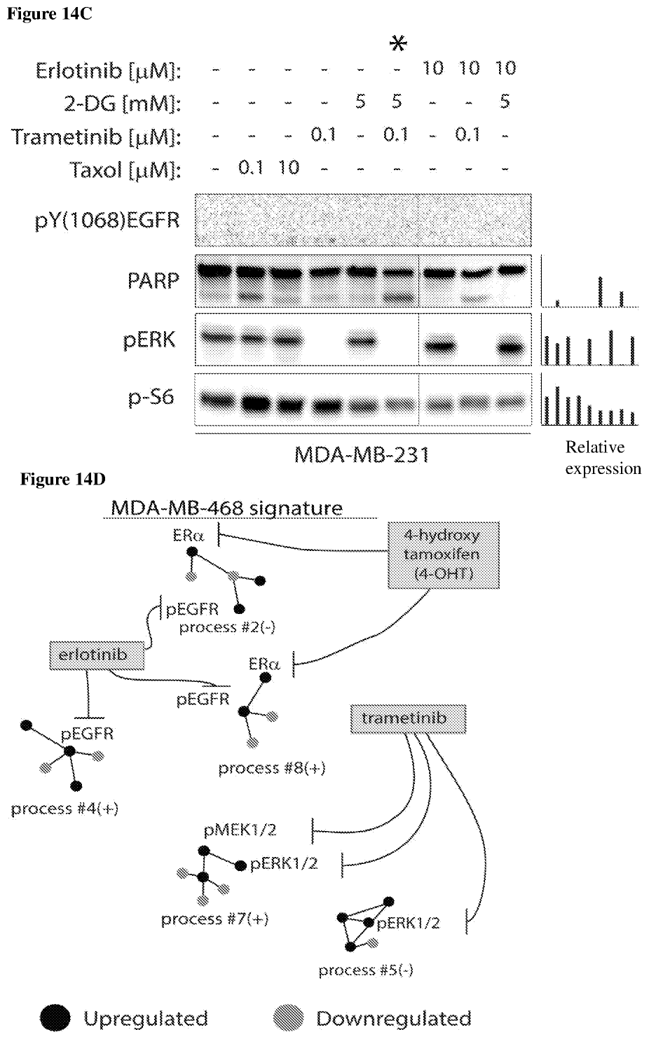

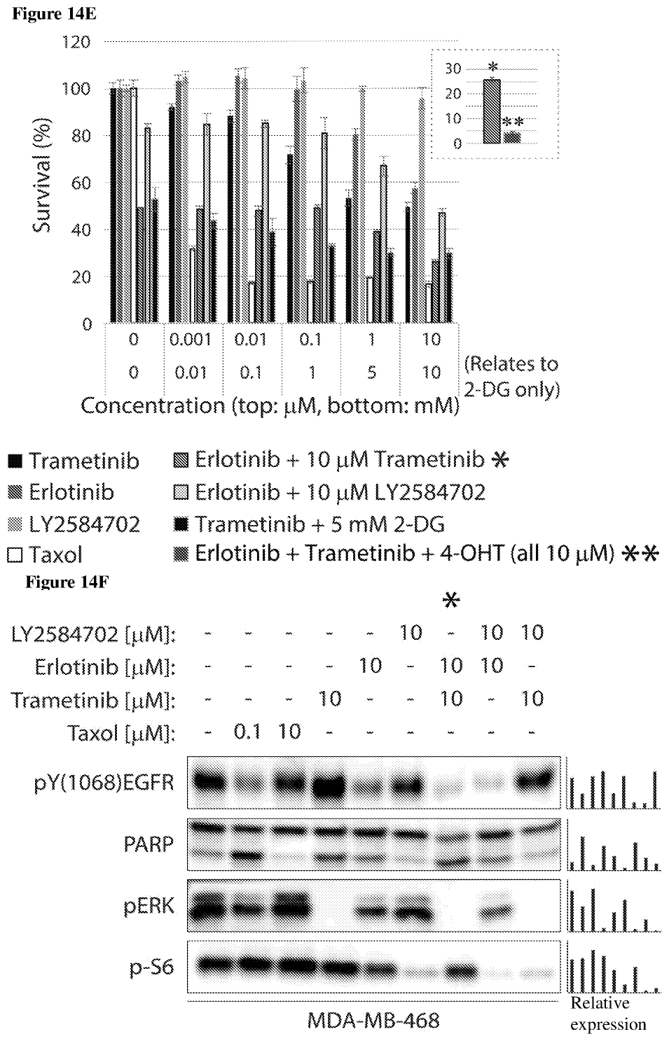

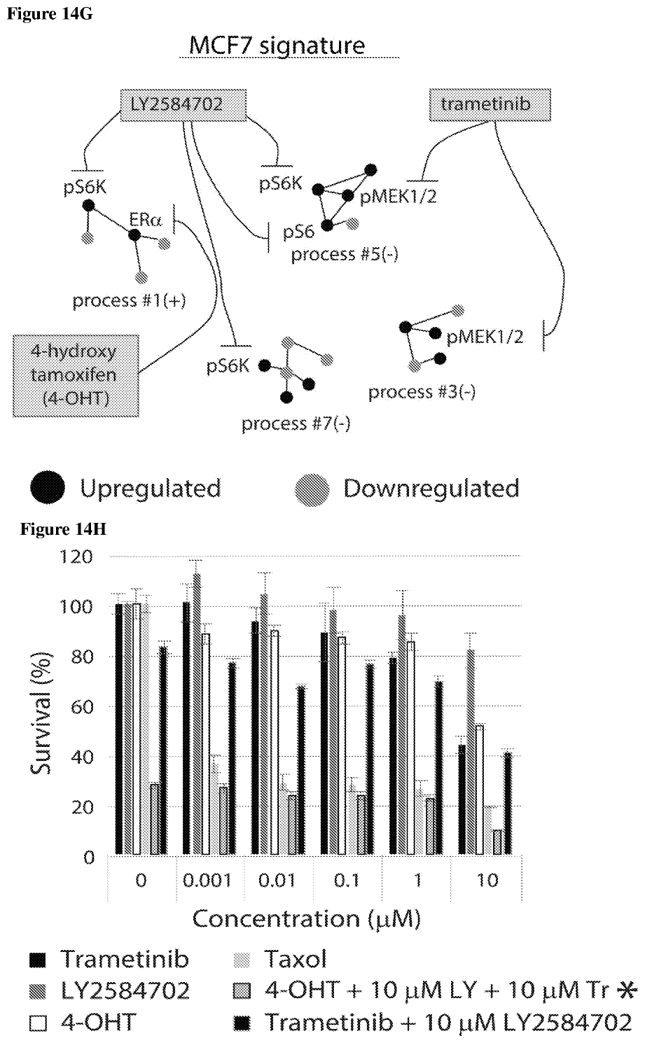

To validate the approach, we analyzed a dataset comprising RPPA measurements from 10 cell lines, including breast, ovarian, and oesophageal cancer, and assigned a barcode and drug combinations to each cell line. The predictions were made such that for each cell line, at least one protein hub from every active unbalanced process will be inhibited, aiming to collapse the sample-specific altered signaling network. Three breast cancer cell lines were chosen for experimental validation: MDA-MB-231 (MD231), MDA-MB-468 (MD468), and MCF7. The two formers represent triple negative breast cancer (TNBC) against which no targeted therapy exists in clinics today. TNBC is often treated with non-specific chemotherapy, such as taxol¹. MCF7 represent luminal type A breast cancer, routinely treated with the ERα inhibitor, tamoxifen, and in some cases chemotherapy such as taxanes. MDA231 cells were indeed efficiently killed by taxol treatment (FIG. 14B). However, the efficacy of the treatment plateaued at 100 nM (FIG. 14B). The MEK1/2 inhibitor, trametinib, T, was predicted to be partially effective, because the MAPK pathway participated in the imbalance in these cells (FIG. 14A). T, however, was not expected to collapse the entire set of unbalanced process in the cells, and indeed a plateau was reached at 100 nM (FIG. 14B). We predicted that to efficiently kill MDA231 cells, T should be combined with the glycolysis inhibitor, 2-deoxy-D-glucose (2-DG) (FIG. 14B). Indeed, the combination was found to be exceptionally effective, and more efficacious than each inhibitor alone or taxol (FIG. 14B). The combination also brought about the cleavage of PARP (FIG. 14C), suggesting apoptotic death. A lower intensity of cleaved PARP was observed when each inhibitor was applied alone (FIG. 14C). In contrast, the EGFR inhibitor, erlotinib, showed no effect on the survival of these cells, alone or when combined with trametinib (FIG. 14C). This is because EGFR was not found to participate in the signaling imbalance in these cells. For MDA468 cells we initially predicted a double combination consisting of T and erlotinib, E (FIG. 14D). The combination killed up to ˜75% of the cells, comparable to the rate of killing achieved by taxol (˜80%), and more effective than each drug alone (FIG. 14E). The combination treatment also induced PARP cleavage (FIG. 14F). We then postulated that since the set of unbalanced processes of MDA468 cells consisted of 5 unbalanced processes (FIG. 14D), and the processes 2, 4 and 8 had at least 2-fold higher amplitudes than in other cell lines, these cells may require an additional drug that will enhance the inhibition of the imbalance. Therefore, we tested the survival of the cells when treated with a triple combination in which the ERα inhibitor, 4-hydroxy tamoxifen (4-OHT; the activated derivative of tamoxifen) was added to T and E. 4-OHT was predicted to target processes 2 and 8, thereby supporting the actions of E in these cells (FIG. 14D). Indeed, the triple combination induced near complete killing of MDA468 cells (FIG. 14E, inset). We tested the combination predicted for MDA231 cells as well, T and 2-DG, showing that it was much less effective in the killing of MDA468 cells, as expected by our analysis (FIG. 14B, 14E).

For MCF7 cells we predicted that using 4-OHT alone (as would be suggested for these cells based on the ERα biomarker) would only partially inhibit the unbalanced signaling flux in these cells, targeting one out of four unbalanced processes that are active in these cells (FIG. 14G). Indeed, 4-OHT killed only up to 50% of the cells (FIG. 14H). We predicted that to collapse the entire signaling flux in these cells, a triple combination, consisting of T, LY2484702, LY (an inhibitor of p70S6K), and 4-OHT, would be more effective (FIG. 14G). FIG. 11H shows that the triple combination was indeed highly effective, surpassing each inhibitor alone, taxol, or the combination of T and LY without 4-OHT. Similar to the case in MDA468 cells, the double combination consisting of T and LY was expected to target all unbalanced processes in these cells, however since there are 4 distinct unbalanced processes that need to be targeted, addition of a third drug is apparently necessary to enhance the effect (FIG. 14G-I). These results coincide with the results obtained in western blot, showing that the triple combination achieved the most significant PARP cleavage (FIG. 14I).

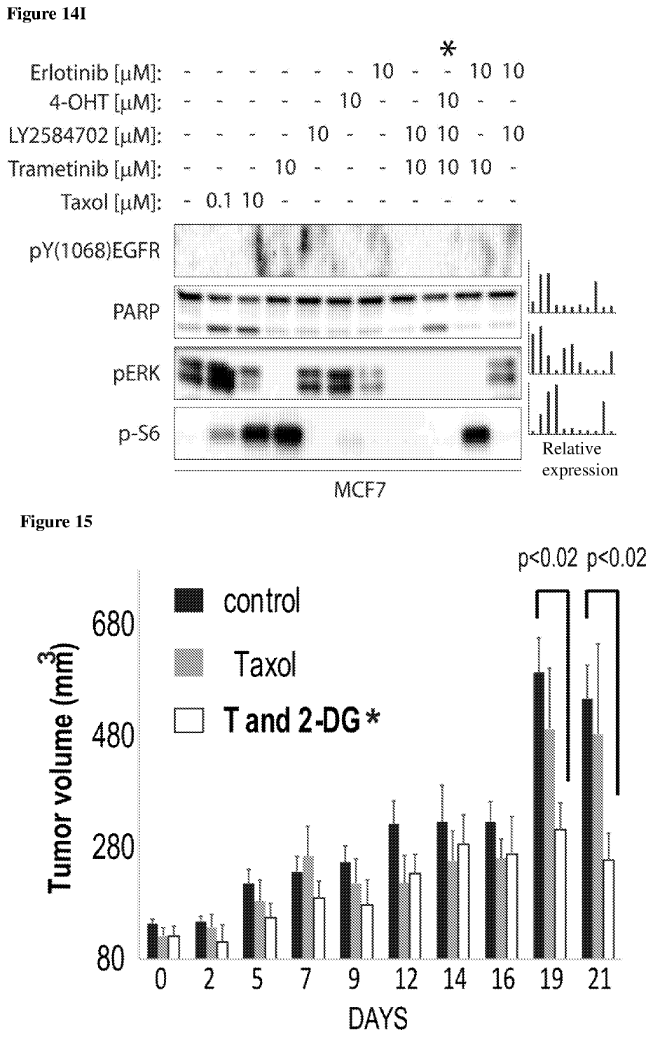

The results were validated in vivo for breast and lung cancer types (see FIG. 15 for experimental details). As shown in FIG. 15, the drug combinations predicted by our analysis, Trametinib and 2-DG (T and 2-DG), work more efficiently than the clinically prescribed Taxol.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method of identifying a druggable target, in a subject suffering from cancer, the method comprising,
a. receiving expression data from said subject;
b. adding said subject's expression data to a first composite cancer-expression data set to produce a second composite cancer-expression data set;
c. determining within said second composite cancer-expression data set at least one unbalanced process, wherein said determining comprises performing thermodynamic-based analysis;
d. identifying within said subject's expression data at least one of said at least one unbalanced processes within said second composite cancer-expression data set; and
e. selecting at least one gene and/or protein from said at least one unbalanced process within said subject's expression data for which a drug that targets said gene or protein is known;
thereby identifying a druggable target in a subject suffering from cancer.

2. The method of claim 1, wherein said expression data is protein expression data or mRNA expression data.

3. The method of claim 1, wherein said receiving expression data comprises receiving a biological sample from said subject and performing high-throughput sequencing on said sample.

4. The method of claim 3, wherein said biological sample is a blood sample or a tumor biopsy.

5. The method of claim 1, further comprising normalizing said subject's expression data with a composite healthy-expression data set or with a composite healthy and cancer-expression data set.

6. The method of claim 1, wherein determining at least one unbalanced process comprises determining over and under expressed genes and/or proteins as compared to their expression in a balanced process.

7. The method of claim 1, wherein determining at least one unbalanced process comprises assembling expressed genes and/or proteins within said second data set into networks.

8. The method of claim 7, wherein said assembling is performed using functional interactions according to the STRING database.

9. The method of claim 1, wherein said thermodynamic-based analysis comprises surprisal analysis.

10. The method of claim 1, wherein said first composite cancer-expression data set comprises data from at least 1 type of cancer.

11. The method of claim 10, wherein said different types of cancer are selected from lymphoma, bladder cancer, gastric cancer, colorectal cancer, kidney cancer, ovarian cancer, endometrial cancer, lung cancer, head and neck cancer, brain cancer and breast cancer.

12. The method of claim 1, wherein said first composite cancer-expression data set comprises data from at least 10 samples.

13. The method of claim 1, wherein said selected at least one unbalanced process is selected from Table 1.

14. The method of claim 1, wherein said at least one gene or protein is over or under expressed in said subject's expression data.

15. The method of claim 1, wherein said at least one gene or protein is a known cancer regulatory gene or protein.

16. The method of claim 1, wherein said at least one gene or protein is selected from Table 1 and Table 3.

17. The method of claim 1, further comprising administering to said subject said known drug.

18. A method for patient-specific cancer treatment, the method comprising,
a. identifying at least one druggable target specific to said patient using the method of claim 1; and
b. administering to said subject at least one drug that targets said at least one druggable target,
thereby providing patient-specific cancer treatment.

19. The method of claim 18, further comprising repeating the method of claim 1 after a period of treatment with said at least one drug to determine at least one new druggable target.

20. (canceled)

21. (canceled)

22. A computer program product for identifying a druggable target, in a subject suffering from cancer, comprising a non-transitory computer-readable storage medium having program code embodied thereon, the program code executable by at least one hardware processor to
c. receive expression data from said subject;
d. add said subject's expression data to a first composite cancer-expression data set to produce a second composite cancer-expression data set;
e. determine within said second composite cancer-expression data set at least one unbalanced processes, wherein said determining comprises performing thermodynamic-based analysis;
f. identify within said subject's expression data at least one of said at least one unbalanced processes within said second composite cancer-expression data set; and
g. provide an output of at least one gene and/or protein from said at least one unbalanced process within said subject's expression data for which a drug that targets said gene or protein is known.
